(19) United States
(12) Patent Application Publication
(10) Pub. No.: US 2008/0206890 A1
(43) Pub. Date: Aug. 28, 2008

(54) BIS(2,9-DI-TERT-BUTYL-1,10-PHENANTHROLINE)COPPER(I) COMPLEXES, METHODS OF SYNTHESIS, AND USES THEROF

(75) Inventors: Judith N. Burstyn, Madison, WI (US); Omar Green, New York City, NY (US); Bhavesh A. Gandhi, Wilmington, DE (US)

Correspondence Address: QUARLES & BRADY LLP, 411 E. WISCONSIN AVENUE, SUITE 2040, MILWAUKEE, WI, 53202-4497, US

(73) Assignee: WISCONSIN ALUMNI RESEARCH FOUNDATION, Madison, WI (US)

(21) Appl. No.: 11/831,533

(22) Filed: Jul. 31, 2007

Related U.S. Application Data
(60) Provisional application No. 60/820,932, filed on Jul. 31, 2006.

(51) Int. Cl. C07F 1/08; G01N 33/553; H01L 31/04

(52) U.S. Cl. 436/525; 546/10; 136/252

(57) ABSTRACT

The present invention provides homoleptic and heteroleptic copper(I) complexes having sterically demanding ligands, such as the bis(2,9-di-tert-butyl-1,10-phenanthroline)copper(I) complex and related complexes, methods of synthesis of these complexes and uses thereof. These copper(I) complexes are useful for various applications including photovoltaic cells, light-emitting electrochemical cells, and analyte sensor systems.

### CROSS-REFERENCE TO RELATED APPLICATIONS

This application claims the benefit of U.S. Provisional Application No. 60/820,932, filed Jul. 31, 2006, which is incorporated by reference herein in its entirety for all purposes.

### FIELD OF THE INVENTION

The invention relates generally to copper(I) complexes and relates specifically to bis(2,9-di-tert-butyl-1,10-phenanthroline)copper(I) complexes, related complexes, methods of synthesis of these complexes and uses thereof.

### BACKGROUND

Copper (I) has been used extensively in the synthesis of molecular machines, bio-inspired model complexes, and industrial catalysts. Many of these applications rely on the geometric reorganization of copper during redox processes, since the ideal geometry is tetrahedral for copper (I) (hereafter Cu(I)) and square planar or tetragonal for copper (II) (Cu(II)). However, Cu(I) complexes are challenging to synthesize due to the intrinsic instability of the cuprous oxidation state. Under many conditions disproportionation of two Cu(I) ions into solid copper and Cu(II) is thermodynamically favored. Nevertheless, the ligand structure and solution conditions may be tailored to favor the formation of Cu(I) complexes.

Complexes of the formula [L₂Cu]⁺, where L is a 2,9-disubstituted-phenanthroline, display interesting photophysical properties due to the metal to ligand charge transfer (MLCT) transition. In the excited state, the copper atom is formally in the +2 oxidation state, and relaxation occurs either through non-emissive geometric reorganization toward square planar geometry or through radiative emission. Therefore, by maximizing the steric bulk of the substituents at the 2 and 9 positions of the phenanthroline ligand while retaining two bidentate phenanthroline ligands on the metal center, maximum radiative emission is achieved by preventing the non-emissive geometric reorganization path.

These [L₂Cu]⁺ complexes have potential applications as inexpensive and environmentally-benign solar energy conversion devices or sensors. In this class of compounds, the homoleptic complexes [(dnpp)₂Cu]⁺ and [(dsbp)₂Cu]⁺ and the heteroleptic complex [(dtbp)(dmp)Cu]⁺ employ the sterically-bulkiest substituents at the 2 and 9 positions of the phenanthroline ligand. These bulky complexes demonstrate the most useful photophysical properties, i.e. long excited-state lifetimes and quantum efficiencies. However, Cu(I) complexes with one phenanthroline ligand and bulky auxiliary ligands such as triphenylphosphine and bis[2-(diphenylphosphino)phenyl]ether have also been shown to exhibit excellent photophysical properties. To optimize the excited-state lifetimes and quantum efficiencies, the considerable synthetic challenge of incorporating highly bulky ligands into Cu(I) complexes must be overcome.

The most common method for the synthesis of Cu(I) complexes has been adding the desired ligand to [Cu(NCCH₃)₄]Y (Equation 1), where Y is PF₆⁻, ClO₄⁻, SbF₆⁻, BF₄⁻, or SO₃CF₃⁻.

[Cu(NCCH₃)₄]Y+nL→[LₙCu]Y+4 CH₃CN (1)

This synthetic method has been successfully used to prepare many sterically-congested Cu(I) systems, including both homoleptic and heteroleptic bis(phenanthroline)-Cu(I) complexes.

Displacement reactions have been used to synthesize homoleptic complexes [Cu(phen)₂]⁺, [Cu(dmp)₂]⁺, [Cu(dpp)₂]⁺, [Cu(bcp)₂]⁺, and [Cu(bfP)₂]⁺. However, larger substituents at the 2 and 9 positions of phenanthroline, i.e. tert-butyl, impair the ability of a second dtbp ligand to compete effectively with acetonitrile for coordination with Cu(I), rendering the formation of [Cu(dtbp)₂]⁺ impossible. Further, adding a less bulky phenanthroline ligand, i.e. dmp, to the (dtbp)Cu(I) complex allows for the synthesis of the heteroleptic complex [(dtbp)(dmp)Cu]⁺.

Other commonly used synthetic methods for preparing Cu(I) complexes include reduction of Cu(II), comproportionation and metathesis (Equation 4). Reducing Cu(II) starting materials by L-ascorbic acid in the presence of ligands in water/alcohol solutions (Equation 2) is often used in the syntheses of Cu(I) phenanthroline complexes. Some Cu(I) complexes are prepared by the comproportionation of Cu(II) and Cu(s) in the presence of an appropriate ligand (Equation 3). Since the formation of Cu(I) from Cu(II) and Cu(s) is unfavorable, these methods rely heavily on the ability of the ligand to stabilize the Cu(I) ion.

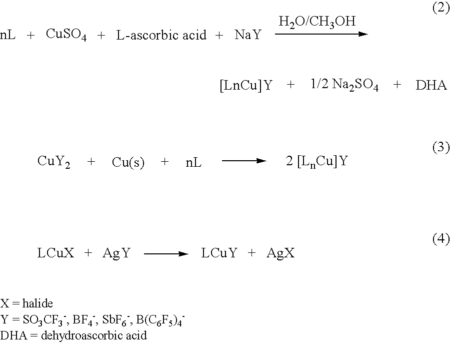

Certain difficult to obtain Cu(I) complexes are predicted to exhibit desirable photophysical properties which result from the metal to ligand charge transfer (MLCT) transition. These complexes have applications as, for example, inexpensive and environmentally-benign solar energy conversion devices or analyte sensors. Accordingly, it is highly desirable in the field to determine new synthetic routes to obtain novel Cu(I) complexes having sterically complex ligands and structures which have industrially useful photophysical properties.

### SUMMARY OF THE INVENTION

The present invention provides improved homoleptic and heteroleptic copper(I) complexes having sterically demanding ligands, such as the bis(2,9-di-tert-butyl-1,10-phenanthroline)copper(I) complex and related complexes, methods of synthesis of these complexes and uses thereof.

In one embodiment, the present invention provides a copper(I) complex having the formula L₁L₂CuX, where X is a negatively charged ion and wherein the ligands L₁ and L₂ are selected from 2,9-di-tert-butyl-4,7-diphenyl-1,10-phenanthroline and 2,9-di-2,4,6-trimethylphenyl-1,10-phenanthroline. The negatively charged ion is preferably selected from SO₃CF₃⁻, BF₄⁻, SbF₆⁻, B(C₆F₅)₄⁻, PF₆⁻ and ClO₄⁻.

In another embodiment, a method of a synthesizing a homoleptic copper(I) complex is provided. The method comprises the steps of: (a) mixing a ligand L and AgX in a molar ratio of about 2:1 and solid copper in a polar solvent to result in a (L)₂CuX complex; and (b) separating the (L)₂CuX complex from the reaction of step (a), wherein X is a negatively charged ion. The polar solvent is selected from acetone, ethanol and tetrahydrofuran (THF). The ion X is selected from the group consisting of SO₃CF₃⁻, BF₄⁻, SbF₆⁻, B(C₆F₅)₄^{-PF}₆⁻ and ClO₄⁻. L comprises any bulky ligand whose ligand to copper(I) ratio is about 2:1, and is challenging to synthesize. In one embodiment, L is selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline.

In another embodiment, a method of a synthesizing a heteroleptic copper(I) complex is provided. The method comprises the steps of: (a) mixing a ligand L₁ with AgX in a molar ratio of at least 1:1 and solid copper in a polar solvent to result in a L₁CuX complex; (b) isolating the resulting L₁CuX complex; (c) adding one molar equivalent of ligand L₂ in a non-polar solvent, resulting in a L₁L₂CuX complex; and (d) separating the L₁L₂CuX complex from the reaction of step (c), wherein X is a negatively charged ion, preferably selected from SO₃CF₃⁻, BF₄⁻, SbF₆⁻, B(C₆F₅)₄⁻, PF₆⁻ and ClO₄⁻. Preferred polar solvent include acetone, ethanol and THF. L₁ and L₂ comprise any bulky ligand whose ligand to copper(I) ratio is about 1:1, and is challenging to synthesize. In one embodiment, L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline.

Another embodiment of the present invention provides a method of detecting a target molecule in a system having or suspected of having the target molecule. Such a method includes steps of: (a) contacting a luminescent copper(I) L₁L₂CuX complex with the system having or suspected of having the target molecule; (b) binding the target molecule to the copper(I) L₁L₂CuX complex, wherein the target molecule has a binding constant for Cu(I) that is greater than a Cu(I) binding constant possessed by at least one of the ligands L₁ or L₂; and (c) detecting the presence of the target molecule by measuring a reduction or increase in luminescence of the copper(I) complex.

The target molecule is preferably selected from CO, CH₃CN, C₂H₄, CH₃NC, C₂H₂, NO and O₂. L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline. The negatively charged ion is preferably selected from SO₃CF₃⁻, BF₄⁻, SbF₆⁻, B(C₆F₅)₄⁻, PF₆⁻ and ClO₄⁻.

In yet another embodiment, a dye-sensitized photovoltaic cell (DSC) is provided. The DSC comprises a light harvesting unit and a sensitizer, wherein the sensitizer is a copper(I) complex L₁L₂CuX and L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline. X is a negatively charged ion, preferably selected from SO₃CF₃⁻, BF₄⁻, SbF₆⁻, B(C₆F₅)₄⁻, PF₆⁻ and ClO₄⁻, The light harvesting unit is based on and comprises nanoparticulate TiO₂.

In another embodiment, the present invention provides a light-emitting electrochemical cell (LEEC) having an emissive layer. The LEEC comprises a copper(I) complex L₁L₂CuX, wherein L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline. Again, X is a negatively charged ion, preferably selected from SO₃CF₃⁻, BF₄⁻, SbF₆⁻, B(C₆F₅)₄⁻, PF₆⁻ and ClO₄⁻.

Another embodiment of the present invention provides a crystalline form of copper(I) complex having unit cell dimensions of about a=11.8246 Å; b=17.8044 Å; and c=27.1111 Å, a=14.906 Å; b=15.188 Å; and c=16.754 Å, a=14.7039 Å; b=25.883(3) Å, and c=16.7036(16) Å, a=14.75449(9) Å, b=15.1383(9) Å, and c=17.9557(11) Å or a=12.1995(5) Å, b=13.6275(6) Å, and c=14.4027(6) Å. In one embodiment, the crystalline form of copper(I) complex is [(2,9-di-tert-butyl-1,10-phenanthroline)₂Cu][B(C₆F₅)₄].

Other objects, features and advantages of the present invention will become apparent after review of the specification, claims and drawings.

### BRIEF DESCRIPTION OF THE FIGURES

FIG. 1: Packing diagram of the unit cell of [(dtbp)Cu(acetone)][SbF₆] (Complex 3) shown along the b axis with 30% probability thermal ellipsoids. Note the occurrence of π-stacking of the phenanthroline planes of two inverted cations in the center. H atoms have been omitted for clarity.

FIG. 2: Molecular diagram of the [(dtbp)₂Cu][B(C₆F₅)₄].CH₂Cl₂ (Complex 5) formula unit shown with 50% probability thermal ellipsoids. The two dtbp ligands are arranged in a pseudo tetrahedral fashion. H atoms have been omitted for clarity.

FIG. 3: Crystal structure for [(dtbbp)₂Cu][SbF₆]. FIG. 3a: plane x. FIG. 3b: plane y. FIG. 3c: plane z.

FIG. 4: Packing diagram of the unit cell of (dtbp)CuBF₄ (Complex 2) shown along the c axis with 50% probability thermal ellipsoids. Note the occurrence of π-stacking of the phenanthroline planes of two inverted cations. H atoms have been omitted for clarity.

FIG. 5: Packing diagrams of (dtbp)CuSO₃CF₃ (Complex 1) shown with views along the a axis (FIG. 5a), b axis (FIG. 5b), and c axis (FIG. 5c) with 30% probability thermal ellipsoids. It is difficult to observe the pairwise π-stacking of inverted complexes because the space group of this complex is P2₁/n, wherein the complexes are π-stacked diagonally to the entire unit cell.

FIG. 6: Face-on molecular diagram of (dtbp)CuSO₃CF₃ (Complex 1) shown with 50% probability thermal ellipsoids. H atoms have been omitted for clarity.

FIG. 7: Molecular diagram of (dtbp)CuBF₄ (Complex 2) shown with 50% probability thermal ellipsoids. Note the orientation of the tert-butyl methyl groups and the bending of the Cu atom and BF₄⁻ ion away from “posterior” methyl groups.

FIG. 8: Molecular diagram of (dtbp)CuSO₃CF₃ (Complex 1) is shown with 50% probability thermal ellipsoids. A slight twisting of the phenanthroline plane can be seen. H atoms have been omitted for clarity.

FIG. 9: Molecular diagram of the [(dtbp)Cu(acetone)][SbF₆] (Complex 3) shown with 50% probability thermal ellipsoids. The Cu atom and the acetone ligand are out of the plane of the phenanthroline ligand, similar to complex 2. H atoms have been omitted for clarity.

FIG. 10: Molecular diagram of the [(dtbp)₂Cu]⁺ cation of Complex 5 shown with 50% probability thermal ellipsoids. The slight distortion from pseudotetrahedral geometry is illustrated here. One dtbp ligand is tilted down and rotated along the phenanthroline plane. H atoms have been omitted for clarity.

FIG. 11: Molecular diagram of one of the two ion-pairs in the asymmetric unit of [(dtbp)₂Cu][BF₄].CH₂Cl₂ is shown with 50% probability thermal ellipsoids. The structure of the cation is similar to that of complex [(dtbp)₂Cu][B(C₆F₅)₄]. The solvent molecule and H atoms are omitted for clarity.

FIG. 12: Molecular drawing diagram of the [(dtbp)₂Cu]⁺ cation of the complex [(dtbp)₂Cu][SbF₆].CH₂Cl₂ shown with 50% probability thermal ellipsoids. The counter ion and H atoms are omitted for clarity.

FIG. 13: Reaction of Complex 1 with C₂H₄ followed by electronic absorption spectroscopy. A solution of 1 (36 mM in CH₂Cl₂) (-) was exposed to excess C₂H₄ (••••). The C₂H₄ was removed by sparging with Ar until all the solvent evaporated and the volume was restored with fresh CH₂Cl₂ (-). This procedure was repeated. The inset shows a magnification of the MLCT region of the absorption spectrum.

FIG. 14: a) Reaction of complex 1 with ethylene; b) Reaction of [Cu(dtbp)(acetone)][SbF₆] with ethylene to produce [Cu(dtbp)(C₂H₄)][SbF₆] (Complex 2).

FIG. 15: Characterization of complex 2 (•••••) and its C₂D₄ analogue 4 (-) by FT-Raman. The C-D stretching region is shown between 2375-2125 cm⁻¹, the C═C stretching region is shown between 1550-1250 cm⁻¹, and the CH₂ and CD₂ wag and scissor region between 1025-725 cm⁻¹. The coupled C═C stretching and CH₂ scissoring motions of complex 2 appear at 1539 cm⁻¹ and 1279 cm⁻¹. The decoupled C═C stretching motion of complex 4 appears at 1402 and the CD₂ scissoring motion appears at 967 cm⁻¹. The CH₂ wagging motion of Complex 2 appears at 980 or 951 cm⁻¹ and the CD₂ wagging motion of 4 appears at 785 cm⁻¹.

FIG. 16—End-on space-filling diagram of the cation of complex 2. Note the rotation of the tert-butyl groups of the phenanthroline ligand.

FIG. 17: Reaction of Complex 1 with CH₃CN followed by absorption spectroscopy. Electronic absorption spectra of Complex 1 (36 μM in CH₂Cl₂) titrated with CH₃CN (0.2 equivalent aliquots up to one equivalent). The spectra obtained after addition of 0 (10), 1 (12) and 3 (14) equivalents of CH₃CN are highlighted. Inset: the MLCT absorption of Complex 1.

FIG. 18: Electronic absorption spectrum of [Cu(dtbp)(NCCH₃)](PF₆) (36 μM in CH₂Cl₂). The spectrum was fit to three Gaussian peaks with maxima at 279 nm, 309 nm and 325 nm.

FIG. 19: Reaction of Complex 1 with CH₃CN followed by emission spectroscopy. Photoluminescence spectra of Complex 1 (36 μM in CH₂Cl₂) titrated with CH₃CN (0.25 equivalent aliquots up to one equivalent). The spectra obtained after addition of 0 (-), 1 (- -) and 3 (-•-) equivalents of CH₃CN are highlighted. The excitation wavelength was 425 nm.

FIG. 20: Reaction of Complex 1 with CH₃CN monitored by ¹H NMR. Shown are the changes in the aliphatic region upon titration of 1 with CH₃CN. Spectra proceed from bottom to top: Complex 1 in CD₂Cl₂ was titrated with 0-1.5 equivalents of CH₃CN (in CD₂Cl₂) in 0.25 equivalent aliquots. The chemical shifts of the various tert-butyl protons of dtbp are indicated: Complex 1 (*; δ1.21 ppm), [Cu(dtbp)(NCCH₃)]⁺ (υ; δ 1.55 ppm), and dtbp (▪; δ1.73 ppm). The resonance that is initially observed at δ2.43 ppm () is assigned to the methyl group of CH₃CN in [Cu(dtbp)(NCCH₃)]⁺.

FIG. 21: ¹H NMR spectrum of [Cu(dtbp)(NCCH₃)](PF₆) (300 MHz, CD₂Cl₂). Resonances: δ 1.75 (s, 18H, CH₃), δ 2.49 (s, 3H, CH₃CN), δ 7.94 (s, 2H, CH), δ 8.09 (d, ³J_{HH}=8.4 Hz, 2H, CH), δ 8.521 (d, ³J_{HH}=8.4 Hz, 2H, CH).

FIG. 22: Titration of Complex 1 with CH₃CN as monitored by FT-Raman spectroscopy. Spectra show the addition of (a) 0, (b) 0.5, (c) 1.0, (d) 1.5, (e) 2.0, and (f) 3.5 equivalent. of CH₃CN to Complex 1 in CH₂Cl₂ solution. Highlighted vibrations include: ν_{CN} of [Cu(dtbp)(NCCH₃)]⁺ (*; 2283 cm⁻¹); ν_{CN} of free CH₃CN in CH₂Cl₂ solution (; 2253 cm⁻¹); an a₁ vibration of free dtbp (υ; 1404 cm⁻¹), and analogous modes of dtbp in [Cu(dtbp)(NCCH₃)]⁺ (▪; 1424 cm⁻¹) and dtbp in Complex 1 (⋄; 1391 cm⁻¹).

FIG. 23: Schematic of the DSC assembly. The device consists of a glass substrate covered by a conductive transparent electrode (F-doped SnO₂, 20). On top of this a compact TiO₂ layer avoids direct contact between the SnO₂ and the hole conductor. The active layer consists of the nanoporous TiO₂ layer covered by the dye and filled with the hole conductor. The counter electrode is a 30-nm gold electrode, which is evaporated on top of the hole conductor.

FIG. 24: Reactions of Complex 1 and [Cu(dtbp)(acetone)](SbF₆) with CO as followed by solution FT-IR spectroscopy. Left column: FT-IR spectra of a solution of Complex 1 in CH₂Cl₂: (a) in the absence of CO; (b) upon exposure to CO; (c) after removal of solvent in vacuo and addition of fresh solvent. Right column: FT-IR spectra of a solution of [Cu(dtbp)(acetone)](SbF₆) in CH₂Cl₂: (d) in the absence of CO; (e) upon exposure to CO; (f) after removal of solvent in vacuo and addition of fresh solvent; (g) the solution from (f) to which excess dtbp was added, the solvent was removed in vacuo, and fresh solvent was added.

FIG. 25: Reaction of Complex 1 with CO in CH₂Cl₂ solution followed by FT-Raman spectroscopy. Spectra show (a) Complex 1 in the absence of CO, (b) an intermediary stage of ligand displacement of dtbp from Complex 1 by CO (c) complete displacement of dtbp from Complex 1 by CO, (d) independently synthesized [Cu(dtbp)(CO)]SbF₆, (e) dtbp. The vibrations labeled are: *, 1391 cm⁻¹, a₁ mode of dtbp in Complex 1; , 1404 cm⁻¹, a₁ mode of free dtbp; ♦, 1424 cm⁻¹, a₁ mode of dtbp in [Cu(dtbp)(CO)]⁺; ⋄, 2031 cm_{••}⁻¹ν_{C≡O} of [Cu(dtbp)(CO)]⁺.

FIG. 26: Minimal electronic absorption spectral changes are observed upon exposure of Complex 1 to O₂. A solution of Complex 1 (36 μM in CH₂Cl₂) (10) was exposed to excess O₂ (12). The O₂ was removed by sparging with Ar (14) followed by a second exposure to excess O₂ (16). Finally, the O₂ was removed again with an Ar sparge (18). The inset shows a magnification of the MLCT region of the absorption spectrum.

FIG. 27: Reaction of Complex 1 with CH₃NC followed by absorption spectroscopy. Electronic absorption spectra of Complex 1 (36 μM in CH₂Cl₂) of Complex 1 titrated with CH₃NC. The spectra obtained after addition of 0 (10), Complex 1 (12) and 6 (14) equivalents of CH₃NC are shown. Inset: the MLCT absorption of Complex 1.

FIG. 28: Schematic drawing showing the currently used embodiment of the Dye-Sensitized Photovoltaic Cells (DSC) utilizing cis-Ru(SCN)₂L₂ (L=2,2′-bipyridyl-4,4′-dicarboxylate) as the dye.

FIG. 29: The cyclic voltammogram of Complex 1 (0.1 M Complex 1 in CH₂Cl₂ with 0.1 M tetrabutylammonium hexafluorophosphate as the supporting electrolyte) measured at 50 mV s⁻¹ with a Ag|AgCl reference electrode, a Pt wire auxiliary electrode and a glassy carbon working electrode. The measured E_{1/2} is 1200 mV vs. Ag|AgCl. The Fc^{0/+} midpoint potential was 500 mV under the same conditions; the calculated E_{1/2} of Complex 1 is 700 mV vs. Fc^{0/+}.

FIG. 30: Two distinct routes used to prepare the appended carboxylate functionality with a minimal tether length 2.

FIG. 31: A method of functionalizing a ligand to provide a point of coordinate covalent attachment to the Ti atoms of the substrate.

FIG. 32: Stepwise assembly of dye on the TiO₂ surface, as illustrated schematically for a representative set of ligands.

FIG. 33: Schematic diagram of the stepwise assembly of a sensitizer dye complex on the surface of a TiO₂ electrode. In step 1, the first ligand, L₁, is attached to the electrode surface through a firmly bonded linkage. In the second step, a preassembled metal complex L₂-M is added to generate the dye complex in situ. When the metal complex degenerates with use, it may be replaced by addition of an excess of the L₂-M to regenerate the sensitizer complex.

FIG. 34: Creation of a molecular layer for covalent attachment of L₁. Chemistry for formation of an amine-terminated TiO₂ surface by photochemical reaction (top). FTIR spectrum of a TiO₂ surface after attachment of the protected amine (bottom).

FIG. 35: Absorption, excitation and emission profiles of [Cu(dtbp)₂]⁺ in CH₂Cl₂. The absorption spectrum (a) exhibits an intense π [symbol] π* transition at 275 nm and a weaker MLCT transition centered at 425 nm. The excitation profile (b) is overlaid on the absorption spectrum. Maximal emission intensity at 599 nm correlates with excitation into the MLCT absorption band. The emission spectrum (c) obtained with 425-nm excitation shows a broad emission peak centered at 599 nm.

### DETAILED DESCRIPTION OF THE INVENTION

It is understood that this invention is not limited to the particular methodology, protocols, and reagents described, as these may vary. One of ordinary skill in the art may change methodology, synthetic protocols and reagents as necessary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

As used herein and in the appended claims, the singular forms “a”, “an”, and “the” include plural reference unless the context clearly dictates otherwise. The terms “a” (or “an”), “one or more” and “at least one” can be used interchangeably herein. It is also to be noted that the terms “comprising”, “including”, “characterized by” and “having” can be used interchangeably.

All publications mentioned herein are incorporated herein by reference for the purpose of describing and disclosing the chemicals, instruments, statistical analysis and methodologies which are reported in the publications which might be used in connection with the invention. Nothing herein is to be construed as an admission that the invention is not entitled to antedate such disclosure by virtue of prior invention.

As described herein, “polar solvents” refer to solvents such as 1,4-dioxane, tetrahydrofuran (THF), acetone, acetonitrile, dimethylformamide, dimethyl sulfoxide, acetic acid, n-butanol, isopropanol, n-propanol, ethanol, methanol, formic acid, water and other similar solvents known to one of ordinary skill in the art.

The invention provides a novel synthetic method allowing access to Cu(I) complexes with weakly-coordinated ligands, complexes which may be useful as molecular machines, bio-mimetic models, catalysts, or solar energy conversion devices. Using Ag(I) with as an oxidizing agent for excess Cu(s) (Eq. 5) provides a novel and straightforward path to Cu(I) complexes. In this high-yielding one-pot reaction, the Cu(s) is oxidized to Cu(I), the ligand is ligated to Cu(I), the Cu(I) is charge-balanced with a desirable counter ion, and, conveniently, Ag(s) and the remaining Cu(s) can be filtered for removal. The reaction proceeds much more quickly in a polar solvent such as acetone than in a more non-polar solvent such as CH₂Cl₂; acetone temporarily ligates and stabilizes the newly formed Cu(I) ion, as demonstrated by the crystallographic data for Complex 3 (vide infra). This reaction allows the synthesis of coordinately unsaturated Cu(I) complexes via a simple reaction in which unwanted species are easily separated via precipitation or crystallization. The present method provides a general approach for the preparation of Cu(I) or Cu(II) complexes with a variety of ligands from solid copper using simple, readily available Ag(I) salts as oxidizing agents.

Dtbp(sol)+AgX(sol)+Cu(s)(acetone)→(dtbp)CuX(sol)+Ag(s) (Eq. 5)

where X=BF₄⁻, SbF₆⁻, SO₃CF₃, or B(ArF)₄⁻

In fact, the previously “impossible” [(dtbp)₂Cu]⁺ complex crystallized out when the oxidation-based reaction was performed with AgB(Ar^{F})₄, as shown in the scheme below:

Dtbp(sol)+AgB(ArF)₄(sol)+Cu(s)(acetone)→½[(dtbp)₂Cu][B(ArF)₄](sol)+½Ag(s)+½AgB(ArF)₄(sol) (Eq. 6)

The complex [(dtbp) Cu]⁺ can also be made deliberately via the scheme below:

2dtbp(sol)+AgB(ArF)₄(sol)+Cu(s)→[(dtbp)₂Cu][B(ArF)₄](sol)+Ag(s) (Eq. 7)

Excess Cu(s) was not necessary for the success of these reactions. Rather the small scale of these reactions would have required measuring inconveniently small quantities of solid copper. On an industrial scale, this reaction could be performed in an environmentally-friendly manner, producing no waste: Solid copper could also be used stoichiometrically, recovering solid silver and solvent for the regeneration of starting materials.

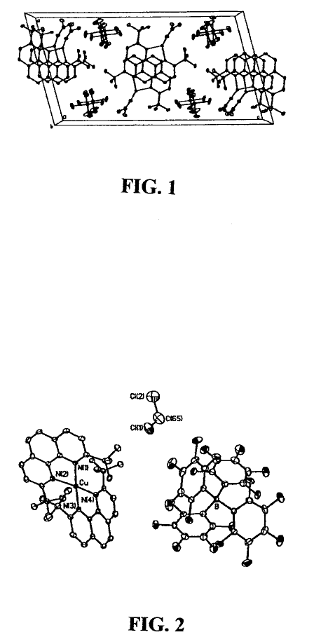

The size and composition of the counterion plays an important role in the structure of the resulting complex, i.e., whether the counterion will bind to the Cu(I) center. The complexes (dtbp)Cu(O₃SCF₃) and (dtbp)Cu(BF₄) comprise bound counterions. In (dtbp)Cu(BF₄), BF₄⁻ is a relatively small counterion and sits comfortably in the cleft created by the tert-butyl groups. As oxygen is a better σ-donor than fluorine, (dtbp)Cu(O₃SCF₃) may be considered to contain the strongest ion-pair, despite the larger size of the O₃SCF₃⁻. The electronic effects out-compete the steric effects. However, as the size of the counterion increases, the open coordination site on the (dtbp)Cu(I) adduct is not large enough to allow for counterion binding, producing a non-coordinating ion pair. As the distance between the metal center and the counterion increases, the stability of the Cu(I) complex decreases, as its coordination sphere is not fully occupied, allowing for the binding of solvent acetone molecules. The crystal structure of Complex 3 (FIG. 1), obtained from a CH₂Cl₂/hexanes solution in poor yield, shows an acetone molecule filling the third coordination site. Since acetone is a ligand to the Cu center, crystallization from acetone/hexanes resulted in significant improvement in the product yield.

Large counterions facilitate the synthesis of [(dtbp)₂Cu]⁺, a complex previously reported to be impossible to prepare. As the size of the counterion is increased from SbF₆⁻ in Complex 3 to B(C₆F₅)₄⁻ in Complexes 4 and 5, the distance between the [(dtbp)Cu]⁺ and the B(C₆F₅)₄⁻ counterion is so great that there is space for a second dtbp ligand to bind to the metal center (FIG. 2). This phenomenon occurred even when only one equivalent of dtbp was added relative to Ag[B(C₆F₅)₄], forming one-half of an equivalent of bis(phenanthroline) complex [(dtbp)₂Cu]⁺. The acetone solution of this complex is yellow, suggesting that the solution state structure is different than the solid state structure. Presumably, crystallization of this compound from acetone/hexanes would produce an acetone adduct, analogous to Complex 3. When an acetone solution of Complex 3 is concentrated, the concentration of dtbp increases relative to the concentration of acetone, and the bidentate dtbp is able to replace the coordinated acetone ligand that is postulated in solution. The resulting orange solid [(dtbp)Cu]B(C₆F₅)₄ forms an orange solution in CH₂Cl₂, indicating that the connectivity of the complex is preserved in non-coordinating solvents.

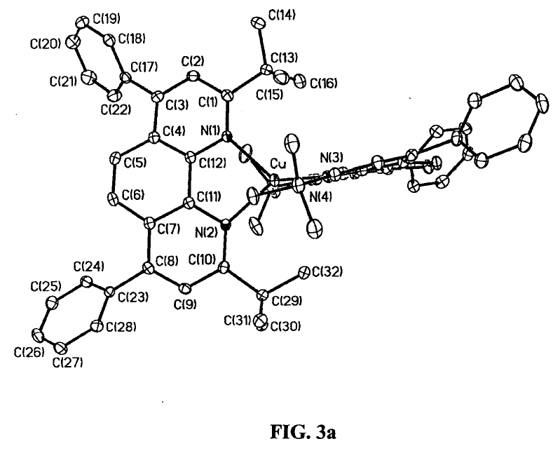

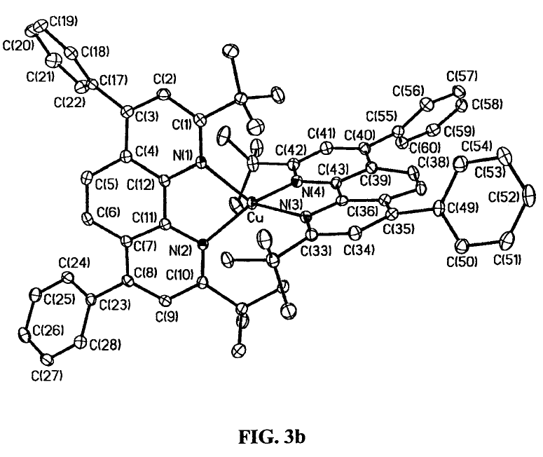

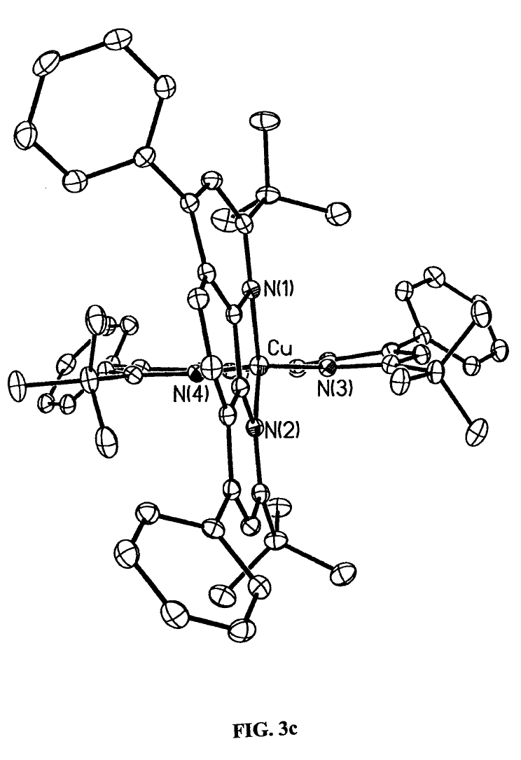

The crystal structures of Complexes 4 and 5 demonstrate particularly long Cu(I)—N distances that are due to steric crowding imposed by the tert-butyl groups of dtbp ligand. The long Cu(I)—N bond distances indicate the difficulty of positioning two dtbp ligands about the Cu center. The average Cu(I)—N distances of Complex 4 (2.112(1) Å) and Complex 5 (2.107(3) Å) are the longest in this class of compounds, longer than the average Cu(I)—N distance of all the bis(phenanthroline)Cu(I) complexes (2.045 Å) found in the Cambridge Structural Database by more than three standard deviations. Crystal structure for [(dtbbp)₂Cu][SbF₆] is shown in FIGS. 3a, 3b and 3c.

At first glance, it may seem that an extraordinarily large counterion like B(C₆F₅)₄⁻ is necessary to prepare the (dtbp)₂ complex. However, once the (dtbp)₂ complex [(dtbp)₂Cu][B(C₆F₅)₄] was synthesized, simple adjustments to the stoichiometry of the reaction, i.e. addition of two equivalents of dtbp relative to AgBF₄ or AgSbF₆ (Eq. 5), allowed for the preparation of Complexes 6 and 7, using smaller counterions and inexpensive, commercially available starting materials. Though considerably smaller than the B(C₆F₅)₄⁻ anion, the SbF₆⁻ anion is sufficiently large to behave in a similar manner to the B(C₆F₅)₄⁻ anion, as it is too large to fit into the cleft created by the two tert-butyl groups of the (dtbp)Cu⁺ moiety, as evidenced by the molecular structure of Complex 3.

The smaller size of BF₄⁻ allows it to compete with free dtbp ligands during reactions for the synthesis of Complex 2. However, when a second equivalent of dtbp ligand is present, there is no competition between the two; dtbp is the better ligand due to the chelate effect and the [(dtbp)₂Cu]BF₄ complex is easily prepared. The fact that a complex that was reportedly impossible to synthesize and which is structurally unfavorable (long Cu(I)—N distances) is the thermodynamically favored product exemplifies the simple beauty of this system.

The [(dtbp)₂Cu]⁺ complex was shown to have superior emission properties as depicted in Table 1. These compounds exhibit highest quantum yield and longest excited state lifetime.

TABLE 1
Emission properties of [(dtbp)₂Cu]⁺ complexes

| | Abs Max, | ε, | Emission | Quantum | |
|---|---|---|---|---|---|
| Complex | Nm | L mol−1 cm−1 | Max, nm | Yield, % | Lifetime, ns |
| [(dtbp)2Cu]+ | 425 | 3100 | 599 | 5 | 3100 |
| [Cu(dmp)(dtbp)]+ | 440 | 7000 | 646 | 1 | 730 |
| [Cu(dmp)2]+ | 454 | 7950 | 730 | 0.023 | 83 |

Accordingly, the present invention provides improved homoleptic and heteroleptic copper(I) complexes having sterically demanding ligands, such as the bis(2,9-di-tert-butyl-1,10-phenanthroline)copper(I) complex and related complexes; methods of synthesis of these complexes and uses thereof.

In one embodiment, the present invention provides a copper(I) complex having the formula L₁L₂CuX, where X is a negatively charged ion and wherein the ligands L₁ and L₂ are selected from 2,9-di-tert-butyl-4,7-diphenyl-1,10-phenanthroline and 2,9-di-2,4,6-trimethylphenyl-1,10-phenanthroline. The negatively charged ion is preferably selected from SO₃CF₃⁻, BF₄⁻, SbF₆⁻, B(C₆F₅)₄⁻, PF₆⁻ and ClO₄⁻.

In another embodiment, a method of a synthesizing a homoleptic copper(I) complex is provided. The method comprises the steps of: (a) mixing a ligand L and AgX in a molar ratio of about 2:1 and solid copper in a polar solvent to result in a (L)₂CuX complex; and (b) separating the (L)₂CuX complex from the reaction of step (a), wherein X is a negatively charged ion. The polar solvent is selected from acetone, ethanol and tetrahydrofuran (THF). The ion X is selected from the group consisting of SO₃CF₃⁻, BF₄⁻, SbF₆⁻, B(C₆F₅)₄ PF₆⁻ and ClO₄⁻. L comprises any bulky ligand whose ligand to copper(I) ratio is about 52:1, and is challenging to synthesize. In one embodiment, L is selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline.

In another embodiment, a method of a synthesizing a heteroleptic copper(I) complex is provided. The method comprises the steps of: (a) mixing a ligand L₁ with AgX in a molar ratio of at least 1:1 and solid copper in a polar solvent to result in a L₁CuX complex; (b) isolating the resulting L₁CuX complex; (c) adding one molar equivalent of ligand L₂ in a non-polar solvent, resulting in a L₁L₂CuX complex; and (d) separating the L₁L₂CuX complex from the reaction of step (c), wherein X is a negatively charged ion, preferably selected from SO₃CF₃⁻, BF₄⁻, SbF₆⁻, B(C₆F₅)₄⁻, PF₆⁻ and ClO₄⁻. Preferred polar solvent include acetone, ethanol and THF. L₁ and L₂ comprise any bulky ligand whose ligand to copper(I) ratio is about 1:1, and is challenging to synthesize. In one embodiment, L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline.

Another embodiment of the present invention provides a method of detecting a target molecule in a system having or suspected of having the target molecule. Such a method includes steps of: (a) contacting a luminescent copper(I) L₁L₂CuX complex with the system having or suspected of having the target molecule; (b) binding the target molecule to the copper(I) L₁L₂CuX complex, wherein the target molecule has a binding constant for Cu(I) that is greater than a Cu(I) binding constant possessed by at least one of the ligands L₁ or L₂; and (c) detecting the presence of the target molecule by measuring a reduction or increase in luminescence of the copper(I) complex.

The target molecule is preferably selected from CO, CH₃CN, C₂H₄, CH₃NC, C₂H₂, NO and O₂. L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline. The negatively charged ion is preferably selected from SO₃CF₃⁻, BF₄, SbF₆⁻, B(C₆F₅)₄⁻, PF₆⁻ and ClO₄.

In yet another embodiment, a dye-sensitized photovoltaic cell (DSC) is provided. The DSC comprises a light harvesting unit and a sensitizer, wherein the sensitizer is a copper(I) complex L₁L₂CuX and L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline. X is a negatively charged ion, preferably selected from SO₃CF₃⁻, BF₄⁻, SbF₆⁻, B(C₆F₅)₄⁻, PF₆⁻ and ClO₄⁻, The light harvesting unit is based on and comprises nanoparticulate TiO₂.

In another embodiment, the present invention provides an organic light-emitting electrochemical cell (OLEEC) having an emissive layer. The OLEEC comprises a copper(I) complex L₁L₂CuX, wherein L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline. Again, X is a negatively charged ion, preferably selected from SO₃CF₃⁻, BF₄⁻, SbF₆⁻, B(C₆F₅)₄⁻, PF₆⁻ and ClO₄⁻.

Another embodiment of the present invention provides a crystalline form of copper(I) complex having unit cell dimensions of about a=11.8246 Å; b=17.8044 Å; and c=27.1111 Å, a=14.906 Å; b=15.188 Å; and c=16.754 Å, a=14.7039 Å; b=25.883(3) Å, and c=16.7036(16) Å, a=14.75449(9) Å, b=15.1383(9) Å, and c=17.9557(11) Å or a=12.1995(5) Å, b=13.6275(6) Å, and c=14.4027(6) Å. In one embodiment, the crystalline form of copper(I) complex is [(2,9-di-tert-butyl-1,10-phenanthroline)₂Cu][B(C₆F₅)₄].

The following examples describe representative synthesis and use of chemical entities according to the invention. These examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Efficient Synthesis of Bulky Cu(I) Complexes

General methods and materials. All chemicals were purchased from Aldrich and used without further purification unless otherwise stated. K(C₆F₅)₄ was purchased from Boulder Scientific Co., copper powder from STREM, and MnO₂ from Fluka; all were used as received. Solvent grade acetone and hexanes were purchased from Columbus Chemical Industries, Inc. and spectrophotometric grade CH₂Cl₂ were from Burdick and Jackson. All copper complex synthesis and crystallization were performed in a glove box under a nitrogen atmosphere. Acetone, dried by distillation from Drierite, and CH₂Cl₂ and hexanes, dried by distillation from calcium hydride, were degassed before use. The ligand, dtbp was synthesized as described previously. AgBF₄ and AgB(C₆F₅)₄ were synthesized as described from AgF and BF₃(CH₃CH₂OCH₂CH₃) and AgNO₃ and KB(C₆F₅)₄, respectively. ¹H and ¹³C NMR spectra were recorded at room temperature (22° C.) on a Varian Mercury-300 MHz spectrometer. Chemical shifts for the spectra were referenced to the residual protons in the deuterated solvent or to the solvent carbons and are reported in parts per million versus Me₄Si. Infrared spectra were recorded on a Bruker Vertex 70 FT-IR spectrometer. Elemental analyses were performed by Desert Analytics.

Synthesis of Cu(dtbp)(CF₃SO₃) (Complex 1). An acetone solution (5 mL) of dtbp (100 mg, 0.342 mmol) was added to a 87.9 mg (0.342 mmol) of AgCF₃SO₃ and an excess of Cu powder (approx. 1 g). The mixture was allowed to stir for 30 minutes. The brown slurry was filtered through CELITE™ and glass wool to remove the black-brown solid. The resulting orange-yellow filtrate was evaporated to dryness under vacuum. The orange solid was dissolved in CH₂Cl₂ and filtered through CELITE™ and glass wool to clarify. Slow evaporation of a CH₂Cl₂/hexanes solution yielded 158 mg (92%) of large (4 mm×4 mm×1 mm) light-orange X-ray quality plates of 1. ¹H NMR (300 MHz, CD₂Cl₂): δ 1.756 (s, 18H, CH₃), δ 7.853 (s, 2H, CH), δ 8.024 (d, J=8.4 Hz, 2H, CH), δ 8.410 (d, J=8.7 Hz, 2H, CH) ppm. ¹³C NMR (75 MHz, CD₂Cl₂) δ 30.70, 38.64, 122.36, 126.15, 127.55, 138.95, 143.72, 170.11 ppm. IR (cm⁻¹): CF₃SO₃⁻, 1234, 1217, 1180, 1161, 1028, 630. Anal Calcd for C₂₁H₂₄N₂CuSO₃F₃: C, 49.94; H, 4.79; N, 5.55. Found: C, 49.58; H, 4.91; N, 5.31.

Synthesis of Cu(dtbp)(BF₄) (Complex 2). An acetone solution (5 mL) of dtbp (100 mg, 0.342 mmol) was added to 66.3 mg (0.342 mmol) of AgBF₄ and an excess of Cu powder (approx. 1 g). The mixture was allowed to stir for 30 minutes. The brown slurry was then filtered through CELITE™ and glass wool to remove a black-brown solid. The resulting yellow filtrate was evaporated to dryness under vacuum. The orange solid was dissolved in CH₂Cl₂ and filtered again to remove any residual solid. Precipitation from CH₂Cl₂/hexanes yielded 143 mg (94.4%) of small thin yellow needles of impure Complex 2. A poor yield of x-ray quality plates was obtained upon slow evaporation of a CH₂Cl₂/hexanes solution. ¹H NMR (300 MHz, CD₂Cl₂): δ 1.731 (s, 18H, CH₃), δ 7.917 (s, 2H, CH), δ 8.064 (d, J=8.7 Hz, 2H, CH), δ 8.479 (d, J=8.4 Hz, 2H, CH) ppm. ¹³C NMR (75 MHz, CD₂Cl₂) ppm. IR (cm⁻¹): BF₄⁻, 1138, 1089, 1054, 1024, 1013.

Synthesis of [Cu(dtbp)((CH₃)₂CO)]SbF₆ (Complex 3). An acetone solution (5 mL) of dtbp (100 mg, 0.342 mmol) was added to 119.6 mg (0.349 mmol) of AgSbF₆ and an excess of Cu powder (approx. 1 g). The mixture was allowed to stir for 1 hr and was then filtered through CELITE™ and glass wool. A black-brown solid was removed, yielding a yellow solution that was concentrated under vacuum. Recrystallization with acetone/hexanes yielded 196 mg (88%) of thin yellow needles of Complex 3. ¹H NMR (300 MHz, CD₂Cl₂): δ 1.677 (s, 18H, CH₃), δ 2.460 (s, 6H, CH₃), δ 7.966 (s, 2H, CH), δ 8.091 (d, J=8.7 Hz, 2H, CH), δ 8.537 (d, J=8.4 Hz, 2H, CH) ppm. ¹³C NMR (75 MHz, CD₂Cl₂) δ 30.64, 32.44, 38.27, 122.60, 126.56, 128.02, 139.91, 143.58, 169.41, 185.99 ppm. IR (cm⁻¹): SbF₆⁻, 654. Anal Calcd for C₂₂H₃₀N₂CuOSbF₆: C, 42.51; H, 4.65; N, 4.31. Found: C, 42.79; H, 4.44; N, 4.15.

Synthesis of [Cu(dtbp)(dmp)]BF₄ (Complex 4). An acetone solution (5 mL) of dtbp (50 mg, 0.171 mmol) was added to 33.1 mg (0.171 mmol) of AgBF₄ and an excess of Cu powder (approx. 1 g). The mixture was allowed to stir for 1 hr and was filtered through CELITE™ and glass wool. A black-brown solid was removed, yielding a yellow solution. Upon addition of 2,9-dimethyl-1,10-phenanthroline (35.6 mg, 0.171 mmol), an intense orange-red color resulted. Crystallization from acetone/hexanes yielded 97.3 mg (87.4%) of orange blocks of Complex 4. The composition was confirmed by comparison with previously reported NMR spectra.

Synthesis of [Cu(dtbp)₂]B(C₆F₅)₄.CH₂Cl₂ (Complex 5). An acetone solution (5 mL) of dtbp (89.6 mg, 0.254 mmol) was added to 100 mg (0.127 mmol) of AgB(C₆F₅)₄ and an excess of Cu powder (approx. 1 g). The mixture was allowed to stir for 30 minutes and was filtered through CELITE™ and glass wool. A black-brown solid was removed, yielding a yellow-orange solution that was then evaporated to dryness under vacuum. The orange solid was dissolved in CH₂Cl₂ and filtered to clarify, producing a clear orange solution. Addition of hexanes followed by slow evaporation yielded 146.3 mg (81.5%) of large bright orange X-ray quality blocks. ¹H NMR (300 MHz, CD₂Cl₂): δ 1.214 (s, 36H, CH₃), δ 7.996 (s, 4H, CH), δ 8.071 (d, J=9.0 Hz, 4H, CH), δ 8.484 (d, J=8.7 Hz, 4H, CH) ppm. ¹³C NMR (75 MHz, CD₂Cl₂) δ 30.69, 39.17, 124.60, 127.55, 129.49, 138.76, 143.61, 169.01 ppm. IR (cm¹): B(C₆F₅)₄⁻, 1511, 1462, 1274, 1087, 979, 774, 768, 756, 683, 660. Anal Calcd for C₆₄H₄₈N₄CuBF₂₀.⅓CH₂Cl₂: C, 56.99; H, 3.62; N, 4.13. Found: C, 57.03; H, 3.83; N, 4.24.

Synthesis of [Cu(dtbp)₂]BF₄.CH₂Cl₂ (Complex 6). An acetone solution (5 mL) of dtbp (100 mg, 0.342 mmol) was added to 33.2 g (0.171 mmol) of AgBF₄ and an excess of Cu powder (approx. 1 g). The mixture was allowed to stir for 30 minutes and then filtered through Celite and glass wool. The resulting clear orange solution was evaporated to dryness under vacuum. The orange solid was dissolved in CH₂Cl₂ and filtered to clarify. Slow evaporation of a CH₂Cl₂/hexanes solution of the resulting orange solution yielded 130.6 mg (93.1%) of large bright needles. ¹H NMR (300 MHz, CD₂Cl₂): δ 1.225 (s, 36H, CH₃), δ 8.043 (s, 4H, CH), δ 8.096 (d, J=8.7 Hz, 4H, CH), δ 8.534 (d, J=8.7 Hz, 4H, CH) ppm. ¹³C NMR (75 MHz, CD₂Cl₂) δ 30.73, 39.13, 124.62, 127.63, 129.60, 138.85, 169.26 ppm. IR (cm⁻¹): BF₄⁻ 1072.73, 1057.27, 1030.78, 633.99. Anal Calcd for C₄₀H₄₈N₄CuBF₄: C, 60.04; H, 6.15; N, 6.83. Found: C, 60.29; H, 6.57; N, 6.70.

Synthesis of [Cu(dtbp)₂]SbF₆.CH₂Cl₂ (Complex 7). An acetone solution (5 mL) of dtbp (100 mg, 0.342 mmol) was added to 59.8 mg (0.171 mmol) of AgSbF₆ and an excess of Cu powder (approx. 1 g). The mixture was allowed to stir for 30 minutes and then filtered through Celite and glass wool. The resulting clear orange solution was evaporated to dryness under vacuum. The orange solid was dissolved in CH₂Cl₂ and filtered to clarify. Slow evaporation of a CH₂Cl₂/hexanes solution of the resulting orange solid yielded 132.4 mg (87.6%) of large bright orange crystals. ¹H NMR (300 MHz, CD₂Cl₂): δ 1.221 (s, 36H, CH₃), δ 8.028 (s, 4H, CH), δ 8.088 (d, J=8.4 Hz, 2H, CH), δ 8.517 (d, J=8.7 Hz, 2H, CH) ppm. ¹³C NMR (75 MHz, CD₂Cl₂) δ 29.74, 36.22, 123.67, 126.45, 128.52, 137.66 ppm. IR (cm⁻¹): SbF₆⁻, 656.32. Anal Calcd for C₂₄H₄₈N₄CuSbF₆.½CH₂Cl₂: C, 52.50; H, 5.33; N, 6.05. Found: C, 52.04; H, 5.39; N, 5.76.

X-ray structure determination. Suitable crystals were selected under oil in air at room temperature. The crystals were mounted on the tip of a nylon loop and immediately placed in stream of nitrogen at 100(2)K. The data collection was performed on a Bruker CCD-1000 diffractometer with Mo K_{α}(λ=0.71073 Å) radiation. The detector was placed at a distance of 4.9 cm from the crystal. The data frames were integrated with the Bruker SAINT-Plus™ software package and corrected for absorption effects using SADABS. Crystal structures for Complexes 1, 2, 3, 4 and 5 were solved by direct methods and all non-hydrogen atoms were identified on the initial electron density map. The non-hydrogen atoms were subsequently refined by full-matrix least-squares methods with anisotropic displacement coefficients. All hydrogen atoms were calculated at idealized positions and were refined as riding atoms with individual isotropic coefficients. Further details of the data collection and refinement are listed in Table 2.

TABLE 2
X-ray crystallographic data of Complexes 1, 2, 3, 5, 6 and 7

| | Complex | | | | | |
|---|---|---|---|---|---|---|
| | 1 | 2 | 3 | 5 | 6 | 7 |
| Formula | C₂₁H₂₄N₂CuSO₃F₃ | C₂₀H₂₄N₂CuBF₄ | C₂₂H₃₀N₂CuOSbF₆ | C₆₅H₅₀N₄CuBF₂₀Cl₂ | C₄₁H₅₀N₄CuBF₄Cl₂ | C₄₁H₅₀N₄CuSbF₆Cl₂ |
| T (K) | 505.2 | 442.76 | 649.78 | 1412.34 | 820.10 | 884.11 |
| λ (Å) | 100(2) | 100(2) | 100(2) | 100(2) | 100(2) | 100(2) |
| Crystal | 0.71073 | 0.71073 | 0.71073 | 0.71073 | 0.71073 | 0.71073 |
| System | | | | | | |
| Space | Monoclinic | Monoclinic | Monoclinic | Monoclinic | Triclinic | Triclinic |
| Group | | | | | | |
| a (Å) | 11.1025(7) | 6.7997(9) | 14.3373(18) | 14.7039(14) | 14.7544(9) | 12.1995(5) |
| b (Å) | 10.3858(7) | 19.943(3) | 6.8138(9) | 25.882(3) | 15.1383(9) | 13.6275(6) |
| c (Å) | 19.1580(12) | 14.0353(18) | 26.391(3) | 16.7036(16) | 17.9557(11) | 14.4027(6) |
| α (°) | 90 | 90 | 90 | 90 | 90.45490(11) | 92.8330(10) |
| β (°) | 97.8480(10) | 9.709(2) | 104.818(2) | 108.057(2) | 91.1900(12) | 109.7370(10) |
| γ (°) | 90 | 90 | 90 | 90 | 98.8550(12) | 107.8140(10) |
| V (Å) | 2188.4(2) | 1901.1(4) | 2492.4(6) | 6043.8(10) | 3961.3(4) | 2114.31(15) |
| Z | 4 | 4 | 4 | 4 | 4 | 2 |
| Dcalc | 1.533 | 1.547 | 1.732 | 1.552 | 1.375 | 1.389 |
| (g/cm³) | | | | | | |
| μ(mm−1) | 1.144 | 1.194 | 2.000 | 0.588 | 0.740 | 1.199 |
| F(000) | 1040 | 912 | 1296 | 2864 | 1712 | 900 |
| Crystal | 0.34 × 0.22 × 0.14 | 0.32 × 0.12 × 0.11 | 0.40 × 0.17 × 0.08 | 0.25 × 0.20 × 0.10 | 0.45 × 0.45 × 0.09 | 0.18 × 0.15 × 0.11 |
| Size | | | | | | |
| (mm³) | | | | | | |
| R1, wR2 | 0.0309, 0.0787 | 0.0489, 0.1170 | 0.0389, 0.0913 | 0.0552, 0.1274 | 0.0539, 0.1425 | 0.0315, 0.0753 |
| ([>(2σI)] | | | | | | |
| R1, wR2 | 0.0375, 0.0827 | 0.683, 0.1278 | 0.0514, 0.0976 | 0.0882, 0.1447 | 0.0637, 0.1489 | 0.10398, 0.0785 |
| all data | | | | | | |

Under the microscope, several seemingly perfect crystals of Complex 6, all of which showed strong reflections with no evidence of twinning, were selected under oil in air at room temperature. Bruker's SMART™ software was unable to determine a unit cell after matrix collections for any of the crystals. After a full data collection of one crystal, CellNow™ was executed, finding a two part twinned crystal. The two domains were rotated 180.0 degrees in 53.6% and 47.4% proportions. The data frames were integrated with the Bruker SAINT-Plus™ software package and the unit cell parameters obtained from CellNow™ and corrected for absorption effects using TWINABS™. The solution and refinement were carried out as described for 1-4.CH₂Cl₂ once the corrected reflection file was obtained.

Two partially occupied solvated molecules of CH₂Cl₂ were present in the asymmetric unit of Complex 7. Bond length restraints were applied to model the molecules but the resulting isotropic displacement coefficients suggested the molecules were mobile. Option SQUEEZE™ of program PLATON™ was used to correct the diffraction data for diffuse scattering effects and to identify the solvate molecule. PLATON™ calculated the upper limit of volume that can be occupied by the solvent to be 366.1 Å³, or 17.3% of the unit cell volume. The program calculated 90 electrons in the unit cell for the diffuse species. This approximately corresponds to two solvate CH₂Cl₂ molecules in the unit cell (84 electrons). It is very likely that this solvate molecule is disordered over several positions. Please note that all derived results in the following tables are based on the known contents. No data are given for the diffusely scattering species.

Facile synthesis of Cu(I) complexes with hindered phenanthroline ligands. Controlled oxidation of metallic copper by stoichiometric amounts of silver salt in the presence of ligand results in the facile synthesis of Cu(I) complexes of hindered phenanthrolines. When excess copper metal is stirred with one equivalent of a Ag(I) salt, and one or two equivalents of 2,9-di-tert-butyl-1,10-phenathroline (dtbp), Cu(I) complexes bearing either one or two bulky phenanthroline ligands may be isolated in high yield (Equation 8). Reaction progress may be easily monitored by eye; reduction of the Ag(I) is observable via formation of a black Ag(s) precipitate, while oxidation of the Cu(s) results in formation of the yellow or orange Cu(I)LₙY (n=1 or 2) species.

Cu(s)+AgY+nL→(acetone)→[LₙCu]Y+Ag(s) (Eq. 8)

This method provides a facile route to synthesize (dtbp)Cu(I)Y complexes, where Y⁻ is one of a variety of weakly-coordinating anions. Mixtures of AgCF₃SO₃, AgBF₄, or AgSbF₆ with dtbp in a 1:1 molar ratio react with Cu(s) to give the mono-dtbp Cu(I) complexes 1, 2, and 3 (92%, 94%, and 88% yields, respectively). These are solvent-facilitated reactions; the redox process will not occur efficiently unless the solvent is of modest polarity and coordinating ability. In acetone and ethanol the redox reaction is complete in minutes, in THF the reaction occurs over the course of hours, and in dichloromethane the reaction may go to completion in days to weeks.

The method is particularly well-suited to the clean preparation of mixed ligand complexes of the hindered phenanthroline ligand, dtbp. The complex, [Cu(dmp)(dtbp)]⁺, where dmp is 2,9-dimethyl-1,10-phenanthroline, may be isolated in high yield and purity, improving on a prior synthesis. Reaction of one equivalent AgBF₄ and one equivalent of dtbp in the presence of excess Cu(s) in acetone produces [(dtbp)Cu((CH₃)₂CO)BF₄ (Complex 12). Direct addition of one equivalent of dmp to the yellow Complex 12 solution resulted in the desired deep orange complex [Cu(dmp)(dtbp)]BF₄ in 87% yield. The NMR of this product showed no evidence of [Cu(dmp)₂]⁺, an impurity reported in the prior synthesis. The order of ligand addition and the steric bulk of dtbp are key to the success of this method: when one equivalent AgCF₃SO₃ and one equivalent of dmp were mixed together and allowed to react with excess Cu(s) the only product was half an equivalent of [Cu(dmp)₂](CF₃SO₃).

Remarkably, this oxidation-based method can be used to prepare the heretofore elusive complex cation [(dtbp)₂Cu]⁺. Mixtures of the silver salts AgB(C₆F₅)₄, AgBF₄, or AgSbF₆ with dtbp in a 1:2 ratio react with excess Cu(s) to afford the [Cu(dtbp)₂]⁺ complexes 5, 6 and 7 in good yields (82%, 93%, and 88%, respectively). The process by which the (dtbp)₂Cu(I) cation is formed is complex and highly solvent dependent. The (dtbp)₂Cu(I) product is only formed if the reactions are carried out in acetone, ethanol or THF. In CH₂Cl₂ or toluene no color change is observed on the same time scale. The initial product in successful reactions is the (dtbp)Cu(I) (solvato) complex; the (dtbp)₂Cu(I) species is formed when the solvent is removed. The initial product is pale yellow (similar to acetone solutions of Complex 3) in acetone. The second ligand appears to bind as acetone is removed; the final product forms a bright orange solution in CH₂Cl₂, and yields bright orange crystals upon precipitation with hexanes.

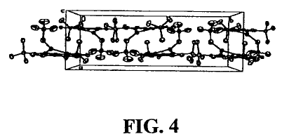

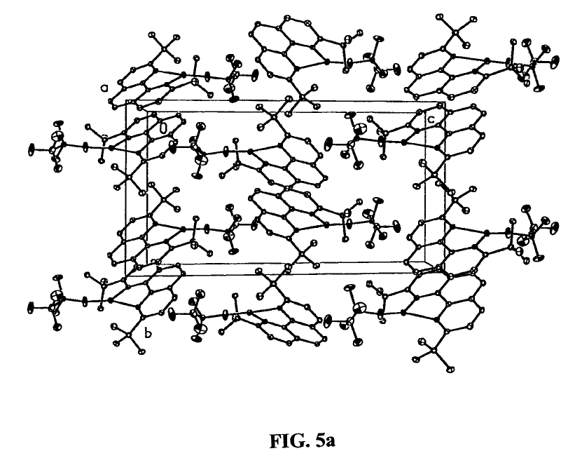

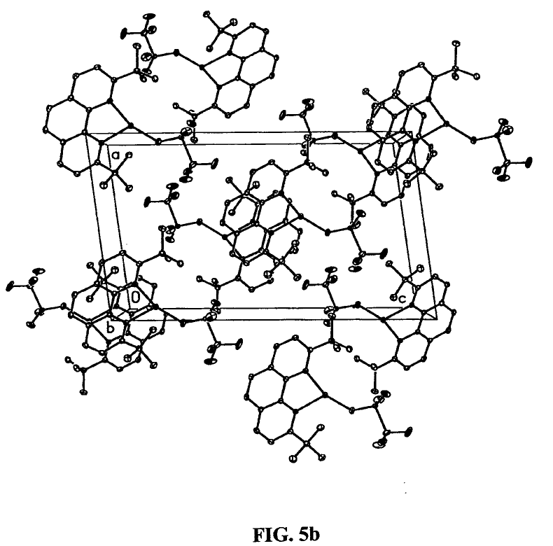

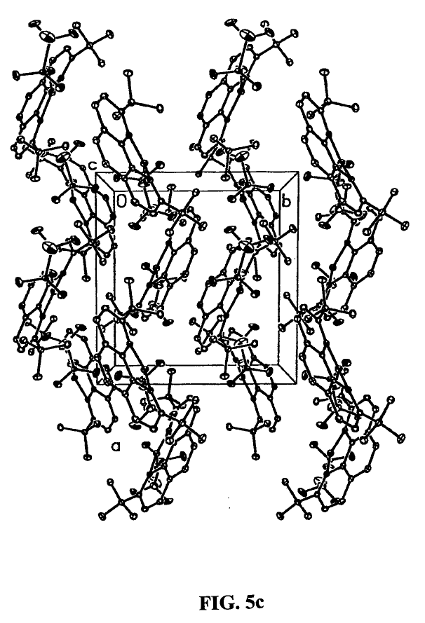

Structures of Cu(dtbp)(CF₃SO₃) (Complex 1) and Cu(dtbp)(BF₄) (Complex 2). The structures of complexes 1 and 2 confirm the 1:1 Cu:dtbp stoichiometry and reveal that the counterion binds to the metal. Complexes 1 and 2 crystallize in the P2₁/n and P2₁/c space groups, respectively, with four formula units occupying each unit cell and no solvent molecules present. The unit cell compositions are thus consistent with a +1 oxidation state for the metal ion. Furthermore, the anomalous scattering properties of the heavy atoms and the elemental analyses are consistent with the lighter Cu atom and not the heavier Ag atom in the complexes. Both compounds participate in π-stacking, but in different ways. The crystal packing of Complex 2, illustrated in FIG. 4, reveals that the phenanthroline rings engage in infinitely-long π-stacking interactions. Between any two stacking molecules lies an inversion center, thus, each molecule is inverted with respect to both of its stacking partners. The phenanthroline planes are stacked at average distances of 3.4 Å and are off-set with respect to one another. The packing diagram of Complex 1, FIGS. 5a, 5b and 5c, reveals that the molecules engage only in pairwise π-stacking interactions. In each pair, the two molecules are off-set and inverted with respect to one another.

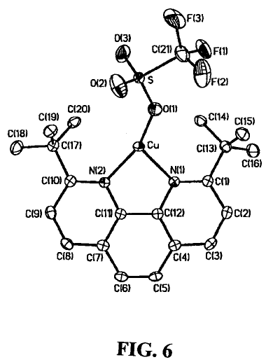

The geometry about the metal ions is distorted trigonal planar. The metal ion in each complex is three coordinate, with the two nitrogens of the phenanthroline (dtbp) and either an O atom (CF₃SO₃⁻) or an F atom (BF₄⁻) as ligands. The coordination environment of the Cu(I) is shown in FIG. 6 for complex 1. Bond lengths are listed in Table 3.

TABLE 3
Significant bond distances of (dtbp)CuOTf (1), (dtbp)CuBF₄ (2),
[(dtbp)Cu((CH₃)₂CO)][SbF₆] (3),
[(dtbp)₂Cu][B(C₆F₅)₄]•CH₂Cl₂(5),
[(dtbp)₂Cu]BF₄•CH₂Cl₂(6),
and [(dtbp)₂Cu]SbF₆•CH₂Cl₂(7), Å

| | 1 | 2 | 3 | 4 | 5 | 6 | | 7 |
|---|---|---|---|---|---|---|---|---|
| Cu—N(1) | 2.1019(16) | 2.040(3) | 2.056(3) | 2.1291(13) | 2.096(3) | 2.081(2) | 2.108(2) | 2.0717(19) |
| Cu—N(2) | 1.9904(15) | 2.019(3) | 2.012(3) | 2.0956(12) | 2.115(3) | 2.105(2) | 2.105(2) | 2.0785(19) |
| Cu—N(3) | | | | 2.1032(12) | 2.076(3) | 2.092(2) | 2.104(2) | 2.145(2) |
| Cu—N(4) | | | | 2.1203(12) | 2.139(3) | 2.114(2) | 2.111(2) | 2.1480(19) |
| Cu—F or O | 1.9272(16) | 2.012(2) | 1.929(3) | | | | | |

| | | | | | | | |
|---|---|---|---|---|---|---|---|
| Avg Cu—N | 2.0462(16) | 2.030(3) | 2.034(3) | 2.1121(12) | 2.107(3) | 2.103(2) | 2.1108(19) |

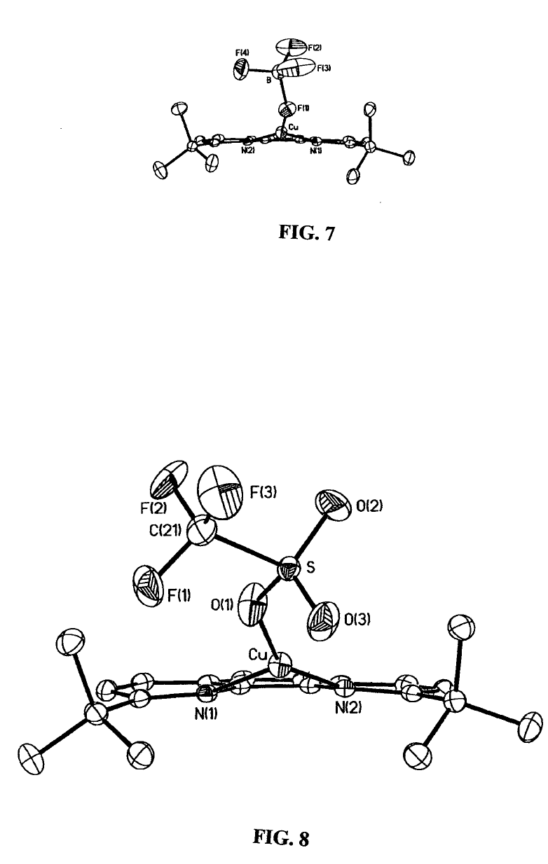

The metal ion and counterion-derived ligand reside above the plane of the phenanthroline, as shown in the packing diagram of Complex 2 in FIG. 4, and when the molecule is viewed along the plane of the phenanthroline ligand in FIG. 7 (Complex 1, FIG. 8). The plane containing the Cu and the two phenanthroline nitrogen atoms is neither coplanar with the phenanthroline aryl rings, nor with the Cu—X (anion atom) bond. The angle between the N—Cu—N plane and the phenanthroline aryl plane is 22.4° in Complex 1 and 14.2° in Complex 2. The angle between the N—Cu—N plane and the Cu—X bond is 16.3° in Cu(dtbp)(CF₃SO₃) and 10.9° in Cu(dtbp)(BF₄). Two pairs of methyl groups point downward, away from the counterion ligand, while the third pair of methyl groups points upward. The counterion is nestled into the cleft created by the upward facing methyl groups. Similar to the previous ¹H NMR observations of dnpp complexes, the chemical shifts of the methyl groups of the substituents of dtbp in Cu(dtbp)(CF₃SO₃) (δ 1.756 ppm) and Cu(dtbp)(BF₄) (δ 1.731 ppm) are shifted downfield with respect to that of free dtbp ligand (δ 1.58 ppm), as expected due to the inductive effect expected upon complexation of the ligand to a Lewis acidic, electropositive center such as Cu(I).

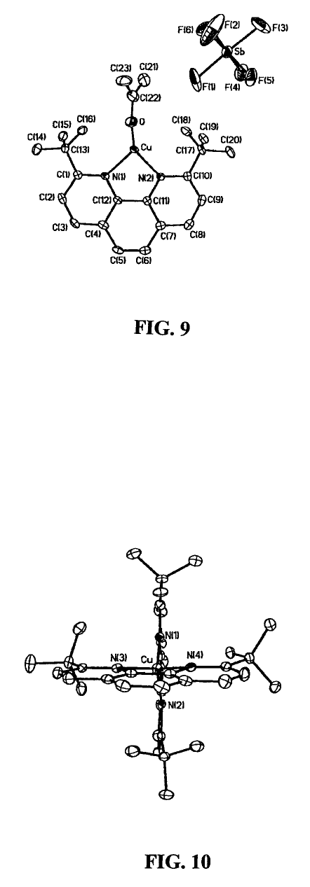

Structure of [Cu(dtbp)(CH₃COCH₃)]SbF₆ (Complex 3). In contrast, in Complex 3 the counterion does not serve as a ligand, rather, a solvent molecule binds to the metal. Complex 3 crystallized in a P2₁/c space group, with four formula units, i.e., four [(dtbp)Cu(acetone)]⁺ cations and four SbF₆⁻ anions, occupying each unit cell. The complex cation is shown in FIG. 9 and the packing diagram with the unit cell is shown in FIG. 1. The anomalous scattering properties of the heavy atom and elemental analysis are only consistent with the presence of a Cu atom in the complex, and the contents of the unit cell are again consistent with the Cu(I) oxidation state of metal ion. Comparison of the packing arrangements between Complex 2 (FIG. 4) and Complex 3 (FIG. 1), which crystallize in the same space group, reveals similar infinite stacking interactions of the Cu-phenanthroline units: the distance between the phenanthroline planes of the two inverted cations in the center of FIG. 1 is 3.4 Å. Interestingly, however, the stacking axes are different between the two crystal structures. In Complex 3, the distance of closest approach between the Cu atom and an F atom of the SbF₆⁻ counterion is 5.072 Å; the shortest distance between the Cu atom and the Sb atom is 6.764 Å.

The coordination sphere of the metal ion in Complex 12 is composed of the two nitrogen atoms of the phenanthroline and the carbonyl oxygen atom of the acetone molecule. The Cu—N(Cu(dtbp)(CF₃SO₃)) and Cu—N(Cu(dtbp)(BF₄)) distances are 2.056(3) Å and 2.012(3) Å, respectively; the Cu(I)—O bond distance is 1.929(3) Å. The coordination sphere of the metal center is distorted trigonal planar, with the metal ion and the acetone ligand above the plane of the phenanthroline. In Complex 12, the angle between the N—Cu—N plane and the phenanthroline aryl plane is 19.6° and the angle between the N—Cu—N plane and the Cu—O bond is 23.1°. Similar to the structures of CF₃SO₃⁻ and BF₄⁻, the methyl groups of the tert-butyl substituents are rotated such that there is only one methyl group is on the face of the phenanthroline where the Cu(I)-acetone moiety resides. As expected, the chemical shift of the methyl group of the dtbp in Complex 12 is also shifted downfield (δ 1.677 ppm) compared to that of free dtbp.

Structures of [Cu(dtbp)₂]B(C₆F₅)₄.CH₂Cl₂ (Complex 5), [Cu(dtbp)₂]BF₄.CH₂Cl₂ (Complex 6), and [Cu(dtbp)₂]SbF₆.CH₂Cl₂ (Complex 7). The previously elusive (dtbp)₂Cu(I) cation is largely similar in structure to other members of this class of compounds, all of which exhibit elongated Cu—N bonds. As observed in the illustration of a single formula unit in FIG. 2, two bulky dtbp ligands coordinate the Cu(I) atom. Crystals of Complex 5 contain four [Cu (dtbp)₂]⁺ cations, four B(C₆F₅)₄⁻ anions, and four CH₂Cl₂ solvent molecules in each unit cell in the P2₁/c space group. The contents of each unit cell are consistent with a +1 oxidation state of the Cu center. Elemental analysis and the anomalous scattering of the heavy atom are consistent with the presence of a Cu atom. The bulky cation and anion are well separated from one another; the distance of closest approach between the Cu atom and an F atom of B(C₆F₅)₄⁻ is 5.500 Å. Interestingly, this is the distance between the Cu atom of one asymmetric unit and the F atom of another asymmetric unit. The distance between the Cu atom and the closest F atom in the same asymmetric unit is 8.466 Å. The shortest distance between the centroids of the cation and anion of different asymmetric units, is 9.416 Å; the distance between the Cu atom and the B atom in the same asymmetric unit is 12.750 Å. Other average Cu—N bond lengths can be seen in Table 4.

TABLE 4
Average Cu—N distances of other bis(phenanthroline) complexes, Å

| Complex | Average Cu—N distance, Å |
|---|---|
| [Cu(dmp)₂]X^{a} | 2.0383 |
| [Cu(dnpp)₂]PF₆ | 2.0622 |
| [Cu(dmp)(dtbp)]PF₆ | 2.0806 |
| [Cu(dpp)₂]X^{a} | 2.0560 |
| [Cu(2,9-C₆F₅-1,10-phen)₂]SbF₆•CH₂Cl₂ | 2.0653 |
| [Cu(xop)₂]PF₆•CH₃OH | 2.0172 |

Dmp = 2,9-dimethyl-1,10-phenanthroline;
dnpp = 2,9-di-neo-pentyl-1,10-phenanthroline;
dpp = 2,9-diphenyl-1,10-phenanthroline;
xop = 2-(2-methylphenyl)-9-(2,6-dimethylphenyl)-1,10-phenanthroline
^{a}Multiple counterions were found in literature, value represents average of all Cu—N distances found.

The coordination geometry of the metal is pseudotetrahedral. The complex exhibits elongated Cu—N bonds resulting in a significant D_{2d} distortion along one axis. The Cu—N(x) (where x=1-4) bond distances are 2.096(3) Å, 2.115(3) Å, 2.076(3) Å, and 2.139(3) Å, respectively, for an average distance of 2.107(3) Å. The intra-ligand N—Cu—N angles are 84.53(10)° and 84.43(10)°, while the inter-ligand N—Cu—N angles are 123.97(10)°, 125.12(10)°, 121.09(10)°, and 122.70(10)°. One phenanthroline ligand is slightly distorted from its position in an idealized D_{2d} geometry.

As illustrated in FIG. 10, the plane of one phenanthroline is tilted slightly downward and displaced to one side. Another crystal form of Complex 4, without a solvent molecule, was obtained; the structure of the cation was similar, though the extent of distortion of the phenanthroline ligand was different. This second structure was of a more idealized D_{2d} cation; minimal distortion of the phenanthroline ligands and a smaller deviation among the Cu—N bond distances were observed (Table 5). The average Cu—N bond length in this second structure was 2.1121(12) Å.

TABLE 5
X-ray Crystallographic Data of [(dtbp)₂Cu]B(C₆F₅)₄ (4),
[dtbpCu(CH₃CN)][PF₆], and [(dtbpCu)₂Cl][SbF₆].

| | 4 | [(dtp)Cu(CH₃CN)][PF₆] | [(dtpCu)₂Cl][SbF₆] |
|---|---|---|---|
| Empirical formula | C₆₄H₄₈N₄CuBF₂₀ | C₂₂H₂₇CuF₆N₃P | C₄₀H₄₈ClCu₂F₆N₄Sb |
| Formula weight | 1327.41 | 541.98 | 983.10 |
| T (K) | 100(2) | 100(2) K | 100(2) K |
| λ (Å) | 0.71073 | 0.71073 Å | 0.71073 Å |
| Crystal system | Monoclinic | Monoclinic | Triclinic |
| Space group | P2₁/c | P2₁/c | P1 |
| a (Å) | 11.8246(5) | 14.3921(7) | 11.4137(5) |
| b (Å) | 17.8044(8) | 15.0667(8) | 14.6454(6) |
| c (Å) | 27.1111(12) | 11.6954(6) | 15.1002(6) |
| [symbol] °) | 90 | 90 | 110.6470(10) |
| [symbol] °) | 90.1670(10) | 112.5200(10) | 94.1180(10) |
| [symbol] °) | 90 | 90 | 111.1270(10) |
| V (Å³) | 5707.7(4) | 2342.7(2) | 2145.27(15) |
| Z | 4 | 4 | 2 |
| D_{calc} (g/cm³) | 1.545 | 1.537 | 1.522 |
| μ (mm⁻¹) | 0.495 | 1.063 | 1.728 |
| F (000) | 2696 | 1112 | 992 |
| Crystal size (mm³) | 0.43 × 0.39 × 0.30 | 0.20 × 0.20 × 0.20 | 0.40 × 0.30 × 0.09 |
| R1, wR2 [I > (2σI)] | 0.0294, 0.0752 | 0.0418, 0.1152 | 0.0355, 0.0890 |
| R1, wR2 (all data) | 0.0365, 0.0797 | 0.0439, 0.1174 | 0.0432, 0.0926 |

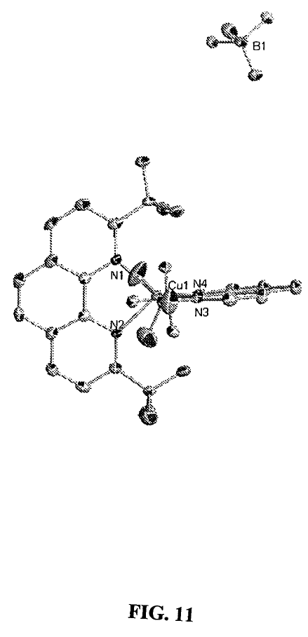

This second form was a less frequently observed morphology for crystals of this compound. The (dtbp)₂Cu(I) cation in Complex 6 is similar to that in Complex 5. The unit cell contains four formula units, i.e. four [Cu (dtbp)₂]⁺ cations, four BF₄⁻ anions, and four CH₂Cl₂ solvent molecules in a P1 space group; the asymmetric unit contains two formula units. The average Cu—N bond distance in the two independent molecules is 2.103(2) Å; individual bond distances are listed in Table 3. The structure of the cation in Complex 62 is similar to the second, less common isomorph of Complex 4, which has a more idealized D_{2d} geometry. The distances between the cations and anions in the structure of Complex 6 are less substantial than in Complex 5, as expected for the smaller anion: Cu—F is 6.292 Å and 7.722 Å; Cu—B is 7.351 Å and 8.804 Å, respectively, for the two molecules in the asymmetric unit (FIG. 11).

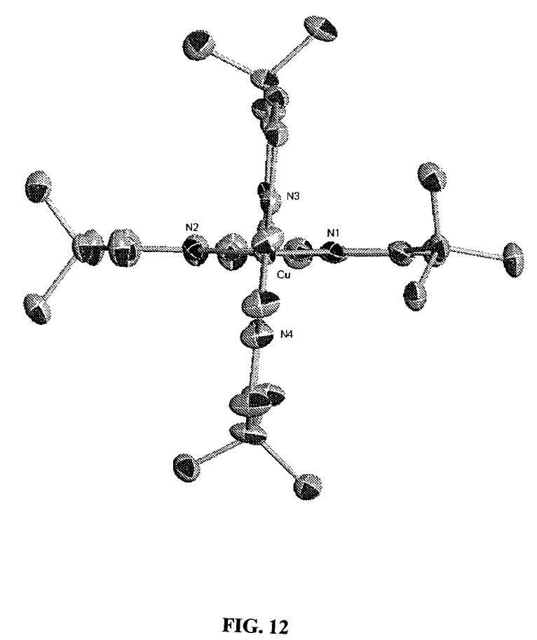

The unit cell of Complex 7 contains two formula units in the P 1̅ space group, i.e, two [Cu(dtbp)₂]⁺ cations, two SbF₆⁻ anions, and two CH₂Cl₂ solvent molecules. The average Cu—N bond distance is 2.111(2) Å, although the individual bond distances (Table 3) span the widest range of the above three complexes. The distortion of the two phenanthroline rings from idealized D_{2d} geometry is also greater than those of the above three complexes. The Cu—F distance is 5.557 Å and the Cu—Sb distance is 7.076 Å. FIG. 12 illustrates the [(dtbp)₂Cu]⁺ cation of 6.CH₂Cl₂. The chemical shifts of the methyl groups of the dtbp ligand in Complexes 5, 6 and 7 (δ 1.214, 1.225, and 1.221 ppm, respectively) are shifted upfield relative to that of free dtbp ligand. This upfield shift phenomenon has previously been attributed to ring current effects on the alkyl groups.

The data and results of this example, including further discussion, is available at Gandhi et al., Inorg. Chem. (2007) 46, 3816-3825, which is incorporated herein by reference.

### Bulky Cu(I) Complexes for Sensing Target Molecules

### A. Ethylene Sensing

Overall, the facile synthetic method allows access to complexes with bulky substituents that were previously inaccessible. This method enabled the synthesis of Complex 4, a complex with interesting optical properties and reactivity due to the size of the tert-butyl substituents. The size of the substituents in the 2 and 9 positions of the phenanthroline ligand has two effects: (a) Minimizing geometric reorganization upon excitation of the MLCT, increasing both the lifetime and the quantum yield; and (b) Providing exogenous ligands access to the ground state Cu(I) center of 4, previously unknown reactivity for bis-phenanthroline-ligated Cu(I) centers.

Synthesis of [Cu(dtbp)₂]SbF₆.CH₂Cl₂ (Complex 1). An acetone solution (5 mL) of dtbp (100 mg, 0.342 mmol) was added to 59.8 mg (0.171 mmol) of AgSbF₆ and an excess of Cu powder (approx. 1 g). The mixture was allowed to stir for 30 minutes and then filtered through Celite and glass wool. The resulting clear orange solution was evaporated to dryness under vacuum. The orange solid was dissolved in CH₂Cl₂ and filtered to clarify. Slow evaporation of a CH₂Cl₂/hexanes solution of the resulting orange solid yielded 132.4 mg (87.6%) of large bright orange crystals. ¹H NMR (300 MHz, CD₂Cl₂): δ 1.221 (s, 36H, CH₃), δ 8.028 (s, 4H, CH), δ 8.088 (d, J=8.4 Hz, 2H, CH), δ 8.517 (d, J=8.7 Hz, 2H, CH) ppm. ¹³C NMR (75 MHz, CD₂Cl₂) δ 29.74, 36.22, 123.67, 126.45, 128.52, 137.66 ppm. IR (cm⁻¹): SbF₆⁻, 656.32. Anal Calcd for C₂₄H₄₈N₄CuSbF₆.½CH₂Cl₂: C, 52.50; H, 5.33; N, 6.05. Found: C, 52.04; H, 5.39; N, 5.76

Synthesis of [Cu(dtbp)(C₂H₄)]SbF₆ (Complex 2). A Schlenk flask charged with yellow [Cu(dtbp)(acetone)](SbF₆) solid was fitted with a stopcock vacuum adapter. Under constant ethylene flow, CH₂Cl₂ was injected into the flask through the outlet stopcock adapter. While stirring, the solution was allowed to evaporate to dryness. The addition of fresh CH₂Cl₂ produced an almost colorless solution. Evaporation of the solvent with C₂H₄ for the second time produced a white solid, but for completeness, an additional third dissolution/evaporation cycle was performed. Crystallization from CH₂Cl₂/hexanes yielded colorless blocks. Complex 2 is air sensitive and care must be taken to prevent contact with adventitious oxygen. Upon exposure to air, the solid acquires a green color. ¹H NMR (300 MHz, CD₂Cl₂): δ 1.695 (s, 18H, CH₃), δ 4.751 (s, 4H, CH₂), δ 8.032 (s, 2H, CH), δ 8.119 (d, J=9 Hz, 2H, CH), δ 8.592 (d, J=9 Hz, 2H, CH) ppm. ¹³C NMR (75 MHz, CD₂Cl₂) δ 30.40, 38.69, 91.44 (C₂H₄), 124.04, 126.81, 128.04, 140.30, 142.69, 171.81 ppm. FT-Raman studies of Complex 2 were performed immediately upon isolation of the compound.

Synthesis of [Cu(dtbp)(C₂D₄)]SbF₆. The C₂D₄ adduct was synthesized directly by exchange from freshly synthesized Complex 2. A CH₂Cl₂ solution of Complex 2 was allowed to stir under an atmosphere of C₂D₄ for 5 minutes, then flushed with Ar to evaporate the solvent. Dissolution, stirring, and flushing were repeated twice to yield a pale yellow solid. FT-Raman studies of the C₂D₄ adduct were performed immediately upon isolation of the compound.

Synthesis of [Cu(dtbp)(CO)]SbF₆. CO was allowed to flow through a vial charged with a bright yellow dichloromethane (CH₂Cl₂) solution of [Cu(dtbp)(acetone)](SbF₆) until the solvent completely evaporated. The resulting solid pale yellow solid was dissolved in fresh CH₂Cl₂ and CO was bubbled again until nothing remained but a white solid. Crystallization of this solid yielded long colorless plates of [Cu(dtbp)(CO)]SbF₆. ¹H NMR (300 MHz, CD₂Cl₂): δ 1.805 (s, 18H, CH₃), δ 8.001 (s, 2H, CH), δ 8.159 (d, J=8.4 Hz, 2H, CH), δ 8.621 (d, J=9 Hz, 2H, CH) ppm. ¹³C NMR (125 MHz, CD₂Cl₂) δ 34.01, 41.29, 125.66, 129.32, 130.72, 143.86, 145.95, 172.72, 173.24 ppm. IR (cm⁻¹): SbF₆⁻, 654; CO, 2130.

Under the microscope, several seemingly perfect crystals of [Cu(dtbp)(CO)]SbF₆, all of which showed strong reflections with no evidence of twinning, were selected under oil in air at room temperature. Bruker's SMART™ software was unable to determine a unit cell after matrix collections for any of the crystals. After a full data collection of one crystal, CellNow™ was executed, finding a two part twinned crystal. The two domains were rotated 179.6° in 53.2% and 47.8% proportions. The data frames were integrated with the Bruker SAINT-Plus™ software package and the unit cell parameters obtained from CellNow™ and corrected for absorption effects using TWINABS™. Crystal structures for [Cu(dtbp)(CO)]SbF₆ were solved by direct methods and all non-hydrogen atoms were identified on the initial electron density map. The non-hydrogen atoms were subsequently refined by full-matrix least-squares methods with anisotropic displacement coefficients. All hydrogen atoms were calculated at idealized positions and were refined as riding atoms with individual isotropic coefficients.

Spectrophotometric studies of ligand reactivity. Electronic absorption spectra were obtained with a Varian Cary 4 Bio spectrophotometer and photoluminescence data were collected with an ISS PC-1 spectrofluorometer outfitted with a 300 W high pressure Xe arc lamp source. All emission spectra were corrected by applying correction factors provided with the instrumental software, which are specific to the instrument. The emission spectrum of Complex 1 in degassed CH₂Cl₂ was recorded using 425 nm excitation. Reaction of Complex 1 with C₂H₄ was also followed by absorption and emission spectroscopy. In these experiments the gaseous ligands (250 μl, 156 equiv) were bubbled into a solution of Complex 1 in CH₂Cl₂ (3.6×10⁻⁵ M) via a gas tight syringe and stirred for 15 min, after which the spectra were recorded. To test the reversibility of these reactions, the solution was then sparged with Ar until dryness, the lost solvent was replaced, and spectra were recorded. This procedure was repeated once more.

X-ray structure determination. A suitable crystal of Complex 2 was selected under oil in air at room temperature. The crystal was mounted on the tip of a nylon loop and immediately placed in stream of nitrogen at 100(2) K. The data collection was performed on a Bruker CCD-1000 diffractometer with Mo K_{α}(λ=0.71073 Å) radiation. The detector was placed at a distance of 4.9 cm from the crystal. The data frames were integrated with the Bruker SAINT-Plus software package and corrected for absorption effects using SADABS. The crystal structure for Complex 2 was solved by direct methods and all non-hydrogen atoms were identified on the initial electron density map. The non-hydrogen atoms and ethylene hydrogen atoms (H21(A, B) and H22(A, B)) were subsequently refined by full-matrix least-squares methods with anisotropic displacement coefficients. All other hydrogen atoms were calculated at idealized positions and were refined as riding atoms with individual isotropic coefficients. Crystal data for [Cu(dtbp)(C₂H₄)]SbF₆: C₂₂H₂₈CuF₆N₂Sb, FW=619.75; monoclinic, space group P2₁/n; a=9.3019(6) Å, b=23.397(2) Å, c=11.6351(7) Å; β=112.278(1)°; V=2343.2(3) Å³; Z=4; D_{calc}=1.757 g/cm³; μ=2.120 mm⁻¹; F(000)=1232; crystal size 0.40×0.40×0.40 mm³; I>(2σI): R₁=0.0167, wR₂=0.0423; all data: R₁=0.0182, wR₂=0.0432.

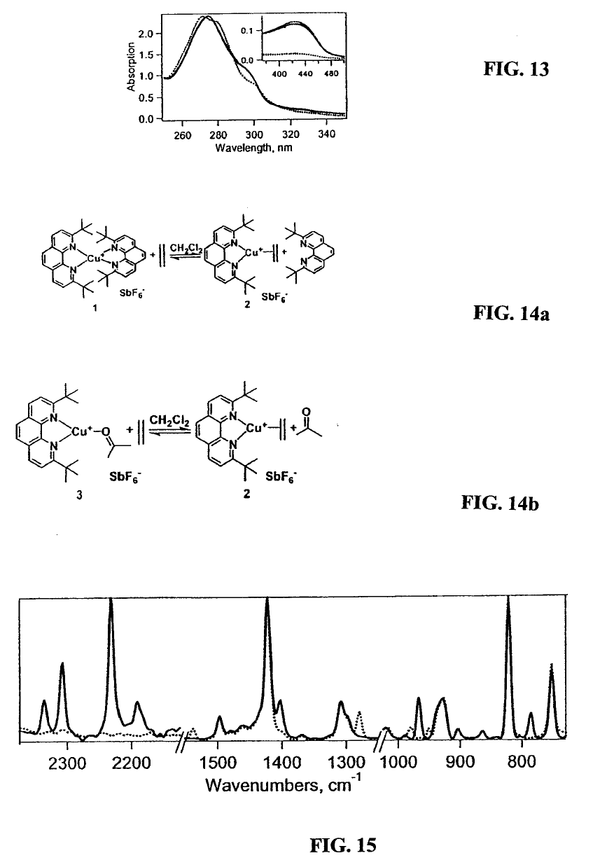

Results. The bis(dtbp) Complex 1 exhibits a metal-to-ligand charge transfer (MLCT) transition at 425 nm (FIG. 13) resulting from the coordination of two phenanthroline ligands on the copper(I) center. Excitation into this MLCT transition band formally produces a copper(II) center in the excited state. The relaxation of the excited state occurs either via geometric reorganization or photoluminescence. The presence of the highly bulky tert-butyl groups inhibits the geometric reorganization pathway and promotes relaxation via the radiative pathway, resulting in high-intensity emission at 599 nm. The bulky tert-butyl groups at the 2 and 9 positions of the phenanthroline ligand rendered the synthesis of Complex 1 impossible via conventional methods. Complex 1 was useful in the sensing of carbon monoxide, as the addition of CO displaced one of the dtbp ligands, forming a [Cu(dtbp)(CO)]⁺ complex. In achieving our goal of sensing ethylene, the observed reactivity of the [Cu(dtbp)₂]⁺ complex, and the existence of the known copper(I)-ethylene complex [Cu(1,10-phenanthroline)(C₂H₄)]⁺ set the stage for the CO-like ethylene-sensing reactivity of complex 1 (FIG. 14a, FIG. 14b).

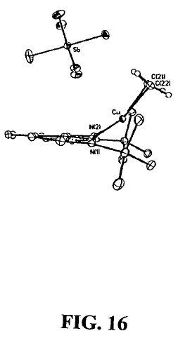

A pale orange solution of Complex 1 (36 μM in CH₂Cl₂) was injected with ethylene gas (250 μL, 156 equivalent), upon which the solution immediately turned colorless. Absorption and emission spectra were recorded after stirring for 15 minutes. The MLCT region of the absorption spectrum (at 425 nm) lost most of its intensity (FIG. 15) and the π-π* region of the spectrum showed a substantial change: the peak at 276 nm split into two peaks at 271 and 280, those of the free dtbp ligand and the presumed mono(dtbp) complex [Cu(dtbp)(C₂H₄)]⁺, respectively. A 36 μM solution of Complex 1 in CH₂Cl₂ was exposed to excess ethylene and allowed to stir for 15 minutes. The gas was removed by sparging with Ar until the solvent was completely evaporated and the solvent volume was restored. This procedure was performed twice.

The emission spectrum of the same sample showed a significant loss of the MLCT-derived emission intensity at 599 nm (Table 6).

TABLE 6
Percent emission of Complex 1 upon exposure to excess ethylene.^{[a]}

| | Emission, % |
|---|---|
| Initial | 100.00 |
| Ethylene exposure 1 | 14.39 |
| Argon sparge 1 | 98.03 |
| Ethylene exposure 2 | 14.11 |
| Argon sparge 2 | 91.37 |

The reversibility of the quenching of the emission was examined by sparging the samples with argon until the solvent was completely evaporated. The sample cells were subsequently refilled with fresh CH₂Cl₂ and the spectra were recorded. The absorption spectra of the Ar-sparged samples show the reverse pattern, wherein the intensity of the MLCT transition at 425 nm is restored and the structure of the π-π* region was re-established. The emission intensity at 599 nm of the Ar-sparged samples was almost completely restored. The data suggest a reversible, inner-sphere sensing phenomenon in which the emission can be almost completely recovered. Complex 1 is so sterically constrained that the presence of a moderately-binding ligand will displace one of the dtbp ligands. In the case of ethylene, the sensing product is presumably the [Cu(dtbp)(C₂H₄)](SbF₆) complex 2.

The ethylene adduct [Cu(dtbp)(C₂H₄)][SbF₆] Complex 2 was independently synthesized from [Cu(dtbp)(acetone)][SbF₆] (Complex 3) and ethylene in CH₂Cl₂. Complex 2 was synthesized by flowing ethylene through a CH₂Cl₂ solution of Complex 3 until the solvent was evaporated to dryness giving a pale yellow solid. The pale yellow color indicates that both Complex 2 and 3 are present. The acetone ligand must be completely removed by evaporation to eliminate the competition for ligation in order to drive the equilibrium toward Complex 3. Thus, the solid was redissolved in CH₂Cl₂ and evaporation under ethylene flow was resumed, ultimately giving a white solid. Here, the lability of copper(I) complexes is a drawback; Complex 2 must be synthesized in this manner because the acetone ligand is a liquid and is readily available to drive the equilibrium in FIG. 14 toward Complex 3. This strategy may be generally useful to synthesize adducts of gaseous ligand when beginning with an adduct of a volatile liquid ligand.

The C₂D₄ analogue [Cu(dtbp)(C₂D₄)][SbF₆] (Complex 4) was synthesized by simply diluting out the C₂H₄ with C₂D₄. A CH₂Cl₂ solution of freshly prepared Complex 3 in a sealed vessel filled with a C₂D₄ atmosphere. Stirring was repeated twice under fresh atmospheres of C₂D₄ to rid the system of C₂H₄. Here, the lability of complex 2 is an advantage, allowing us to use dramatically less C₂D₄ relative to the amount of C₂H₄ used for the synthesis of Complex 2. Because both ligands are gases, there was no need to flow C₂D₄ to complete dryness of the solution. The excess of C₂D₄ (10-15×) in the atmosphere of the flask is enough to statistically exchange a large portion of the C₂H₄ with argon flushing away the displaced C₂H₄ at each step.

Complex 3 was characterized by ¹H and ¹³C NMR, FT-Raman spectroscopy, and X-ray crystallography. The conversion of Complex 3 to Complex 2 was followed by ¹H NMR. ¹H NMR spectrum of the white solid product, complex 2, (in CD₂Cl₂) showed the disappearance of the acetone methyl singlet at δ 2.460 ppm and the appearance of the ethylene singlet at δ 4.751 ppm. A similar pattern was observed in the ¹³C NMR spectrum as the chemical shifts attributed to acetone, 32.44 ppm from methyl and 185.99 ppm from carbonyl, disappear and a new shift attributed to olefin carbons appears at 91.44 ppm.

FT-Raman spectra of complex 2 and its C₂D₄ analogue Complex 4 were collected to corroborate the presence of ethylene. The spectrum of 2 contains two prominent peaks at 1279 and 1539 cm⁻¹, as shown in FIG. 15. The C₂D₄ isotopomer displays a single peak in that region at 1402 cm⁻¹. In addition, the C₂D₄ isotopomer also exhibits peaks at 2191, 2232, 2307, and 2336 cm⁻¹, corresponding to C-D stretches and peaks at 785 and 967 cm⁻¹. Similar observations were reported by Hiraishi for Zeise's salt, K[PtCl₃(C₂H₄)].H₂O, and later by Hirsch et al. for [Cu([9]aneS₃)(C₂H₄)][BF₄]. The peak was assigned at 1402 cm⁻¹ as the decoupled C═C stretching mode and the peak at 967 cm⁻¹ as the decoupled CD₂ scissoring motions in the spectrum of the C₂D₄ adduct.

Complex 2 crystallized in a P2₁/n space group, with four formula units, i.e., four [Cu(dtbp)(C₂H₄)]⁺ cations and four SbF₆— anions, occupying the unit cell. The formula unit is shown in FIG. 16. The scattering properties of the heavy atom are only consistent with the presence of a copper atom in the complex, and the contents of the unit cell are consistent with the copper(I) oxidation state of metal ion. Packing diagrams reveal that the phenanthroline planes of the cations of Complex 2 engage in pair-wise π-stacking with a distance of 3.5 Å. The two cations are inverted with respect to each other with the coordinated ethylene pointing away from the stacked phenanthroline rings. In Complex 2, the distance of closest approach between the Cu atom and an F atom of the SbF₆⁻ counterion is 3.699 Å; the shortest distance between the Cu atom and the Sb atom is 5.394 Å.

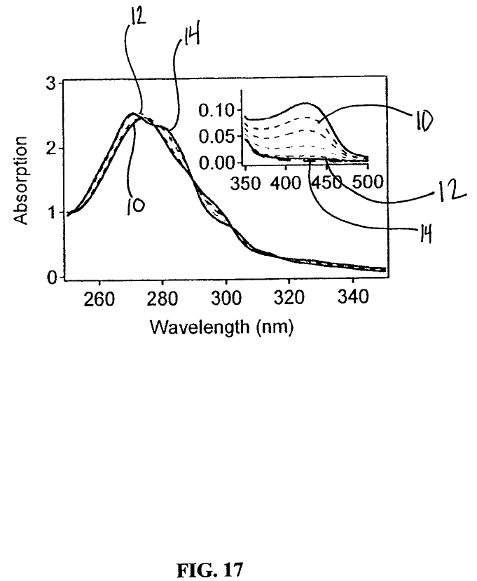

The coordination sphere of the metal ion in Complex 2 is composed of the two nitrogen atoms of the phenanthroline and the carbon atoms of the ethylene molecule. The Cu—N(I) and Cu—N(2) distances are 2.038(1) Å and 2.019(1) Å, respectively; the Cu—C(21) and Cu—C(22) distances are 2.048(2) Å and 2.033(2) Å, respectively. The C(21)-C(22) bond distance is 1.360(3) Å. The coordination sphere of the metal center is distorted trigonal planar (considering the centroid of the ethylene ligand), with the metal ion and the ethylene ligand above the plane of the phenanthroline. In Complex 2, the angle between the N—Cu—N plane and the phenanthroline aryl plane is 33.6° and the angle between the N—Cu—N plane and the C(21)-Cu—C(22) bond is 21.6°. As shown in the space-filling diagrams in FIG. 17, the methyl groups of the tert-butyl substituents are rotated such that there is only one methyl group on the side of the phenanthroline where the copper(I)-ethylene moiety resides.

Crystallographic refinement of the ethylene hydrogen atoms showed bending of these hydrogen atoms away from the copper(I) center. After refinement of the non-hydrogen atoms, the next 28 peaks, with intensities between 0.91 and 0.67 eÅ⁻³, on the difference Fourier map all corresponded to H atoms based on their placement. The next highest peak had an intensity of 0.52 eÅ⁻³. The peaks corresponding to ethylene hydrogen atoms had intensities of 0.87, 0.82, 0.81, and 0.76 eÅ⁻³ and were subsequently refined as ethylene hydrogen atoms. The remaining hydrogen atoms were then calculated at idealized positions. The angles between H(21a)-C(21)-H(21b) and H(22a)-C(22)-H(22b) in ethylene are 115.77° and 115.86°. The angle between the two H—C—H planes of the ethylene ligand is 24.9°. A comparison of the pertinent angles and distances relative to literature values are shown in Table 7.

TABLE 7
Bond distances and angles of complex 2, free ethylene, and other metal-ethylene complexes.

| | Free C₂H₄ | | Other | Other |
|---|---|---|---|---|
| | (calculated)^{[a]} | Complex 2 | copper(I)-C₂H₄^{[b]} | nickel(0)-C₂H₄^{[c]} |
| Distances, Å | | | | |
| M—N | — | 2.038(1), 2.019(1) | 1.972-2.032 | — |
| M—C | — | 2.048(2), 2.033(2) | 1.943-2.028 | 1.957-2.049 |
| C═C | 1.3305(10) | 1.360(3) | 1.346-1.361 | 1.387-1.417 |
| Angles, ° | | | | |
| H—C—H | 121.45(10) | 115.77, 115.86 | — | 111.86-117.12 |
| (H—C—H plane)- | 0 | 24.9 | — | — |
| (H—C—H plane) | | | | |

The bound ethylene ligand in complex 2 is similar to those of other copper(I)-ethylene complexes, such that they all exhibit C═C bond distances very close to that of free ethylene. The ethylene C═C bond distance of 1.360 Å in complex 2 is slightly longer than that of free ethylene calculated recently (1.3305 Å). This small extension of the bond length has also been seen in previously synthesized copper(I)-ethylene complexes, which feature ethylene C═C bond distances between 1.346 Å and 1.361 Å. These C═C distances in copper(I)-ethylene complexes are markedly closer to that of free ethylene than are ethylene C═C distances in the isoelectronic Ni(0)-ethylene complexes, which are between 1.387 and 1.417 Å. Complex 2 is approximately 22° away from trigonal planar geometry about the copper(I) ion, unlike previously reported copper(I)-ethylene complexes, which were shown to be trigonal planar about copper(I). Likely, the steric bulk of the tert-butyl group (FIG. 17) forces this deviation. The crystallographically-refined hydrogen atoms of the ethylene point slightly away (each H—C—H plane is ˜12.5° from planarity) from the copper(I). Unfortunately, there is no such data in the literature for previously reported copper(I)-ethylene complexes with which to compare these data.

Conclusion. The sterically-constrained weak bonding between the Cu(I)—N in the highly luminescent complex [Cu(dtbp)₂]⁺ demonstrates its utility as a molecular ethylene sensor. The identity of the sensing product has been confirmed by independently synthesizing [Cu(dtbp)(C₂H₄)]⁺ (2).

### B. Acetonitrile, CO and O₂ Sensing

Binding affinity of the second dtbp ligand. Complex 1 undergoes facile ligand replacement reactions, where one dtbp ligand is lost. When Complex 1 was dissolved in coordinating solvents, its characteristic bright orange color disappeared and was replaced by a yellow color. This color change occurred in methanol, acetone and CH₃CN, but not in CH₂Cl₂. Since the bright orange color is characteristic of bis(phenanthroline) coordination to copper(I), it appeared that one of the ligands was displaced upon dissolution, even in a weakly coordinating solvent like acetone (Equation 9, Y is solvent).

[Cu(dtbp)₂]⁺+Y [symbol] [Cu(dtbp)(Y)]⁺+dtbp (Eq. 9)

The binding affinity of the second dtbp ligand in Complex 1 was determined by measuring a binding constant for Equation 10. In CH₂Cl₂, Complex 10 was titrated with dtbp, and the growth of the MLCT absorption at 425 nm was monitored. The data were fit to an expression appropriate for complexes with high stability constants; the resulting binding constant for dtbp was (9.9±0.3)×10⁵. This binding constant defines the relative affinities of acetone and dtbp for the Cu center, as it was not possible to prepare a complex bearing one dtbp and no other ligand.

[Cu(dtbp)(acetone)](SbF₆)+dtbp [symbol] [Cu(dtbp)₂](SbF₆)+acetone (Eq. 10)

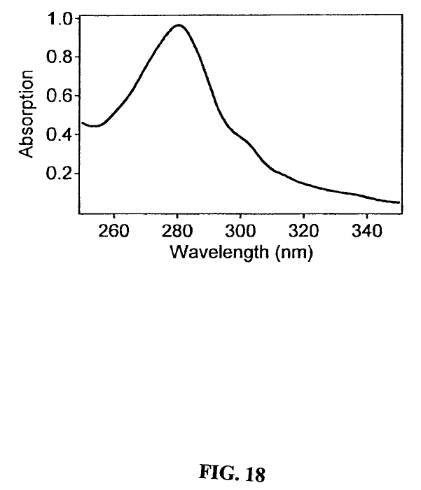

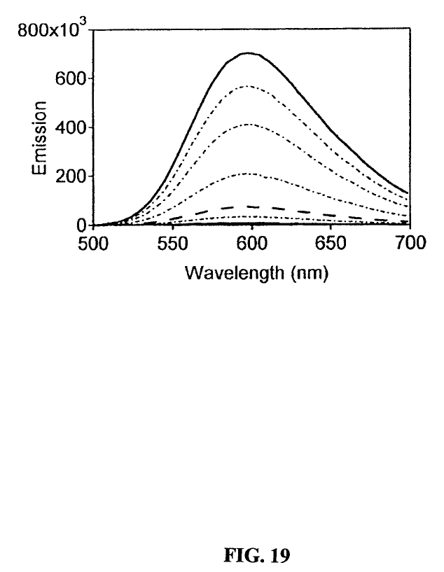

Reactivity of Complex 1 with CH₃CN. The reaction of Complex 1 with CH₃CN was studied in more detail. Changes attributable to the displacement of dtbp from Complex 1 were observed in the absorption and emission spectra of Complex 1 upon titration with CH₃CN (Equation 11, FIG. 17). Most noteworthy was the loss of the MLCT absorption at 425 nm upon addition of 1 equivalent of CH₃CN, and the appearance of new features between 300 to 325 nm. Changes in the π→π* transition region were also observed. As CH₃CN was added, the 275-nm band split into two new bands: one at 271 nm, the position of the π→π* absorption of the free dtbp ligand, and one at 279 nm. Independent synthesis and crystallographic and spectroscopic characterization (FIG. 18) of [Cu(dtbp)(NCCH₃)](PF₆) confirmed that the π→π* absorptions at 279 nm, 310 nm and 325 nm were from the complex [Cu(dtbp)(NCCH₃)]⁺. Addition of CH₃CN to 1 also resulted in loss of the MLCT-derived emission at 599 nm (FIG. 19). Note that in both absorption and emission experiments spectral changes are observed up to one equivalent of CH₃CN; minimal further changes are observed upon addition of up to three equivalents CH₃CN. The equilibrium constant for Equation 11, describing the relative affinities of CH₃CN to dtbp, was calculated to be (4±2)×10⁷.

[Cu(dtbp)₂]⁺+CH₃CN [symbol] [Cu(dtbp)(CH₃CN)]⁺+dtbp (Eq. 11)

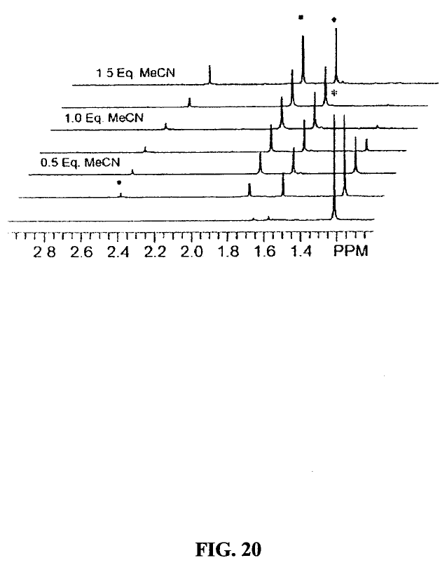

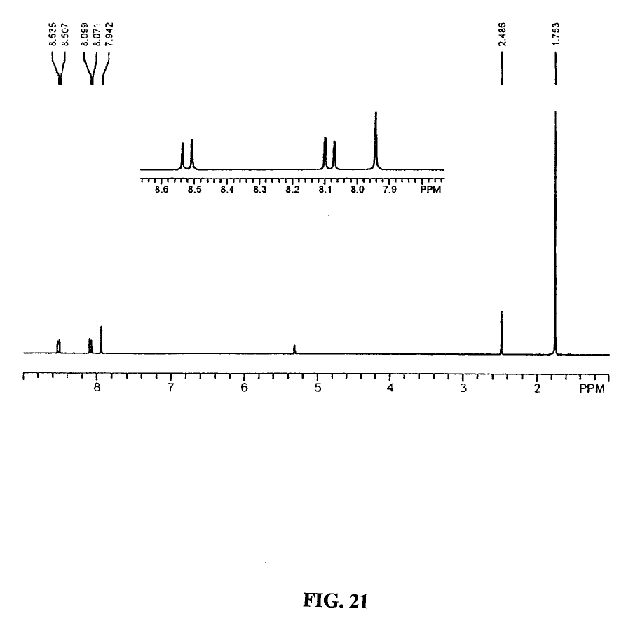

The displacement of dtbp by CH₃CN was explicitly demonstrated by ¹H NMR (FIG. 20), following the distinct resonances of the tert-butyl substituents of dtbp. The tert-butyl groups of 1 appear at 1.21 ppm, the tert-butyl groups of the free dtbp ligand appear at 1.55 ppm, and those of the complex [Cu(dtbp)(NCCH₃)]⁺ appear at 1.75 ppm (FIG. 21). As CH₃CN is added to 1, the intensity of the resonance at 1.21 ppm decreases and new resonances grow in simultaneously at 1.55 ppm and 1.73 ppm. This observation clearly reveals that CH₃CN displaces one dtbp ligand from the coordination sphere of Cu. The reaction is essentially complete upon addition of one equivalent CH₃CN. At one equivalent, the 1.21 ppm resonance of Complex 1 is barely observed. Minimal further change in the intensity of the resonances of free dtbp (1.55 ppm) and [Cu(dtbp)(NCCH₃)]⁺ (1.73 ppm) occurs upon addition of up to three equivalents CH₃CN. The methyl protons of CH₃CN appear at 2.43 ppm in [Cu(dtbp)(NCCH₃)]⁺ as it is formed from Complex 1 upon addition of one equivalent CH₃CN. When excess CH₃CN is added, this resonance shifts toward that of free CH₃CN (2.04 ppm) suggesting that there is rapid exchange between the free and bound CH₃CN.

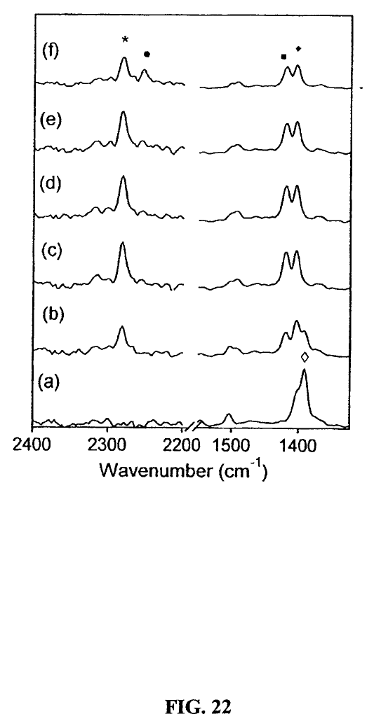

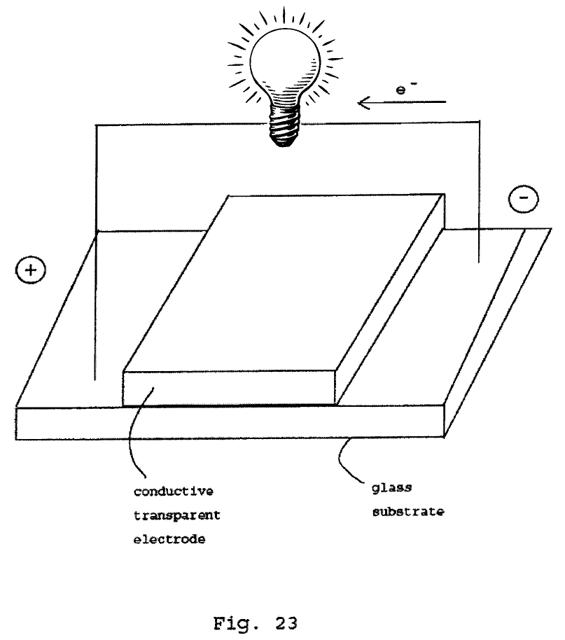

FT Raman analysis corroborates the displacement of a single dtbp ligand by CH₃CN (FIG. 22). In the absence of CH₃CN, the Raman spectrum of Complex 1 revealed a ligand-based ring vibration at 1391 cm⁻¹, with a shoulder at higher energy (FIG. 23). The analogous vibration appears at 1404 cm⁻¹ in free dtbp and at 1420 cm⁻¹ in [Cu(dtbp)(NCCH₃)]⁺. As CH₃CN is added to Complex 1, the peak at 1391 cm⁻¹ is replaced by two peaks, at the positions characteristic of the free ligand (1404 cm⁻¹) and [Cu(dtbp)(NCCH₃)]⁺ (1421 cm⁻¹). The reaction can also be followed by the appearance of the C—N mode of bound CH₃CN in [Cu(dtbp)(NCCH₃)]⁺ at 2283 cm⁻¹: this mode is completely absent in Complex 1 and grows in only as CH₃CN is added. When three equivalents of CH₃CN were added, an additional peak corresponding to free CH₃CN was observed at 2253 cm⁻¹. Notably, the ν_{C≡N} of bound CH₃CN in [Cu(dtbp)(NCCH₃)]⁺ is 30 cm⁻¹ above the value of ν_{C≡N} observed for free CH₃CN, indicating a non-classical interaction of copper(I) with CH₃CN in this complex.

Reactivity of Complex 1 with CO. CO displaces one dtbp ligand from Complex 1 in a reaction analogous to that observed for CH₃CN (Equation 12). The [Cu(dtbp)(CO)]⁺ complex was independently synthesized from [Cu(dtbp)(acetone)]⁺ and the characterization of this new complex will be discussed below as it pertains to the reactivity of Complex 1. Absorption, emission, FT Raman and IR spectroscopies were used to monitor the reaction of Complex 1 with excess CO; the spectroscopic features characteristic of Complex 1 are replaced by those characteristic of [Cu(dtbp)(CO)]⁺ and free dtbp upon reaction. A color change from bright orange to an almost-colorless pale yellow is observed as gaseous CO is added. This color change is complete within seconds. As in the reaction with CH₃CN, the MLCT absorption at 425 nm characteristic of Complex 1 is lost, and the π→π* transition at 275 nm is replaced by two new absorption features at 284 nm and 271 nm, due to [Cu(dtbp)(CO)]⁺ and free dtbp, respectively (FIG. 13). A 36 micromolar solution of Complex 1 in CH₂Cl₂ was exposed to excess CO or O₂. The gas was removed by sparging with Ar and the solvent volume was restored. This procedure was repeated for a total of two times. Loss of the MLCT absorption band of 1 upon reaction with CO results in near complete loss of the MLCT-derived emission at 599 nm (Table 8).

TABLE 8
Excess CO and O₂ reversibly quench the emission of Complex 1.

Percent Initial
Emission (I/Iₒ × 100)

| | CO | O₂ |
|---|---|---|
| 1 in CH₂Cl₂ | 100% | 100% |
| 1^{st} exposure | 3.7% | 44% |
| 1^{st} Ar sparge | 87% | 89% |
| 2^{nd} exposure | 2.9% | 42% |
| 2^{nd} Ar sparge | 70% | 83% |

Also notable are changes in the ligand based ring vibrations in the FT Raman spectrum (FIG. 14), analogous to those observed in reaction with CH₃CN (FIG. 23), with the appearance of a new C—O stretch at 2130 cm⁻¹.

[Cu(dtbp)₂]⁺+CO [symbol] [Cu(dtbp)(CO)]⁺+dtbp (Eq. 12)

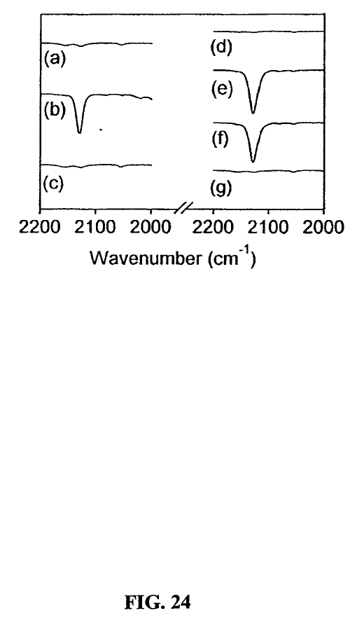

The reaction of Complex 1 with CO is reversible. When the solution obtained after reaction of 1 with CO, which contained [Cu(dtbp)(CO)]⁺ and free dtbp, was sparged with Ar, the characteristic absorption and emission features of Complex 1 returned. When CO was reintroduced, the products [Cu(dtbp)(CO)]⁺ and free dtbp were once again observed. Exposing solutions of Complex 1 to cycles of CO and Ar, with restoration of evaporated solvent, revealed that after two complete cycles (CO exposure, degassing with Ar, addition of lost solvent) 70% of the initial photoluminescence intensity was recovered (Table 8). Evidence of incomplete reversion is present in the absorption spectra; the intensity of the MLCT absorption of Complex 1 was not fully restored after addition and removal of CO. The reversibility of Equation 12 was also studied by FT-IR. A series of spectra were recorded: 1) before exposure of Complex 1 to CO, 2) after bubbling with CO, and 3) after the reaction solution had been taken to dryness in vacuo and the resulting solid redissolved in fresh CH₂Cl₂. FIG. 24 shows the ν_{C≡O} stretching region of these spectra: no ν_{C≡O} band was seen before exposure of 1 to CO (FIG. 24a) but upon exposure of 1 to CO, a C≡O stretch appeared at 2130 cm⁻¹ (FIG. 24b). When the products were dried in vacuo and redissolved in CH₂Cl₂ the C≡O stretch disappeared from the spectrum (FIG. 24c).

Reversible CO binding is only observed when a second dtbp ligand is present. FT-IR studies, parallel to those described for Complex 1 above, were performed using the complex [Cu(dtbp)(acetone)](SbF₆). Excess CO reacted readily with [Cu(dtbp)(acetone)](SbF₆) to form [Cu(dtbp)(CO)]⁺, as illustrated in FIG. 24d and FIG. 24e. When the product [Cu(dtbp)(CO)]⁺ was treated in the same manner as the product of the reaction of Complex 1 with CO, i.e. taken to dryness under vacuum and redissolved in fresh solvent, the CO remained bound to the metal center (FIG. 24f). Excess dtbp ligand was then added to the same solution, the solvent was removed in vacuo, and the resulting solid was redissolved. The solution product in the presence of excess dtbp ligand bore no CO (FIG. 24g). These experiments reveal that reversible CO binding is enabled by the presence of uncoordinated dtbp, which replaces CO as the ligand.

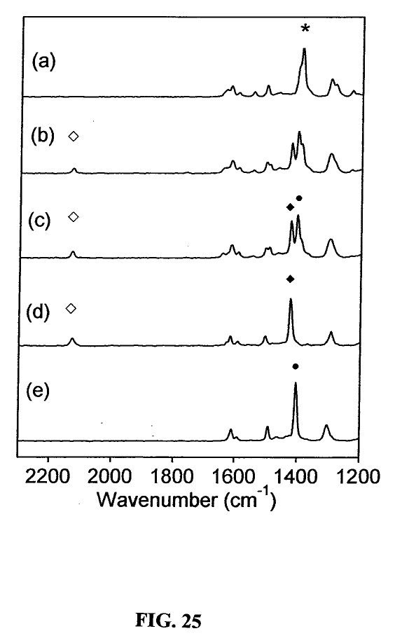

The complex [Cu(dtbp)(CO)]⁺ was independently synthesized in order to corroborate the spectral assignments made in the reaction of Complex 1 with CO. Equation 13 was carried out in the non-coordinating solvent CH₂Cl₂ to prepare [Cu(dtbp)(CO)](SbF₆). Evaporation of the solvent under a CO flow was necessary to completely remove the acetone ligand, thus eliminating any competition for ligation to the copper(I) center. As shown in FIG. 25, the IR spectrum of this complex in CH₂Cl₂ solution reveals a ν_{C≡O} mode at 2130 cm⁻¹, illustrating classical binding of CO to copper(I). The band at 2130 cm⁻¹ shifts to 2080 cm⁻¹ when the solution of [Cu(dtbp)(CO)]⁺ is equilibrated with ¹³CO, thus conclusively identifying this as the ν_{C≡O} mode. The ¹H NMR spectrum of [Cu(dtbp)(CO)](SbF₆) showed no evidence of acetone methyl groups and the tert-butyl methyl proton resonance is shifted downfield relative to that of [Cu(dtbp)(acetone)](SbF₆). The quaternary carbon of the CO ligand appeared at 173.24 ppm in the ¹³C NMR spectrum.

[Cu(dtbp)(acetone)](SbF₆)+CO [symbol] [Cu(dtbp)(CO)](SbF₆)+acetone (Eq. 13)

[Cu(dtbp)(CO)](SbF₆) crystallized in a P 1̅ space group, with two formula units, i.e., two [Cu(dtbp)(CO)]⁺ cations and two SbF₆⁻ anions, occupying the unit cell. The coordination sphere of the metal ion in Complex 2 is composed of the two nitrogen atoms of the phenanthroline and the carbon atom of the CO ligand. The Cu—N(1) and Cu—N(2) distances are 2.045(2) Å and 2.034(3) Å, respectively; the Cu—C(21) distance is 1.814(3) Å. The C(21)-O bond distance is 1.132(3) Å, only slightly longer than the 1.128 Å of free CO. Interestingly, the coordination sphere of the metal center is trigonal planar; this complex is the first three-coordinate [Cu(dtbp)X]^{+/0} complex in which the third ligand lies in the phenanthroline plane. The methyl groups of the tert-butyl substituents are positioned symmetrically so as to limit crowding about the CO ligand. The C₂ᵥ symmetric structure of this complex is in contrast to the distorted asymmetry of previously characterized [Cu(dtbp)X]^{+/0} complexes.

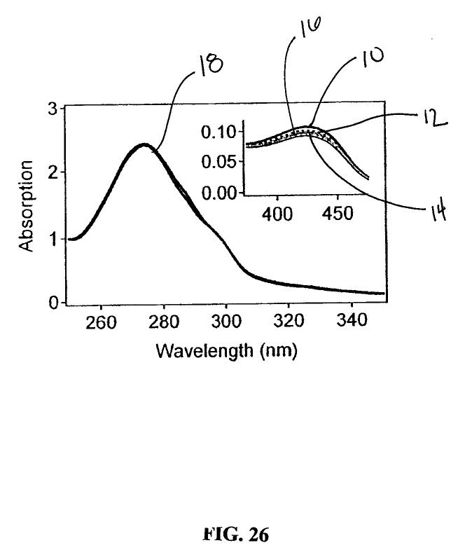

Reactivity of Complex 1 with O₂. Dioxygen does not displace dtbp from Complex 1, but O₂ does partially quench the luminescence of Complex 1. Upon exposure to excess O₂ the absorption spectrum of Complex 1 changes slightly; a minor diminution of the intensity of the π→π* ligand absorption (275 nm) is observed FIG. 26. There is no evidence of an absorption band at 271 nm from the free ligand or of any other new bands. Similarly, there is a modest change in the intensity of the MLCT absorption of Complex 1 upon O₂ addition. Since the MLCT-derived emission intensity is very sensitive to the presence of the second dtbp ligand, we conclude that O₂ does not displace dtbp from Complex 1. Excess O₂ does quench the luminescence of Complex 1, albeit to a substantially lesser extent than excess CO (Table 8). The luminescence quench observed on exposure of Complex 1 to O₂ is reversible upon sparging the solution with Ar. The quenching induced by O₂ is more reversible than that induced by CO; after two exposures of Complex 1 to O₂, each followed by removal with an Ar sparge, 83% of the initial emission intensity is restored.

[Cu(dtbp)₂]⁺+O₂→No Reaction (Eq. 14)

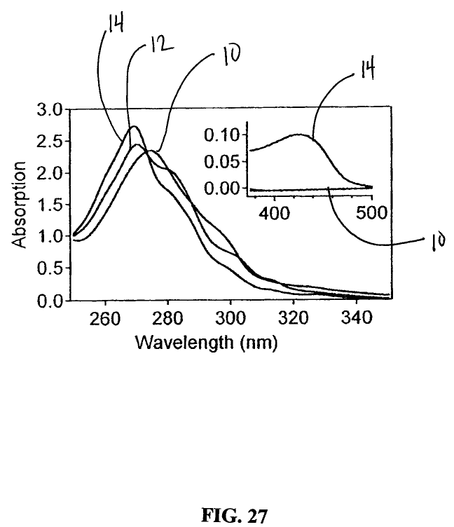

Reactivity of Complex 1 with CH₃NC. CH₃NC displaces both dtbp ligands from Complex 1. As was observed for reaction of Complex 1 with CH₃CN and CO, addition of 1 equivalent CH₃NC is accompanied by changes in the absorption spectrum consistent with displacement of one dtbp ligand from the metal (FIG. 27). However, unlike CH₃CN or CO, addition of CH₃NC beyond 1 equivalent results in a continued increase in the absorption at 271 nm corresponding to free dtbp, indicating that CH₃NC is capable of displacing the second dtbp. Equilibrium constants for reactions 15a and 15b were calculated from the absorption data. The binding constant for the first CH₃NC (Equation 15a) was found to be 3×10⁹ using the intensity at 425 nm from 0-1 equivalent CH₃NC. The binding constant for the additional CH₃NC ligands (Equation 15b) was calculated to be 4×10³ using intensities at 269 nm from 1-6 equivalent CH₃NC. The identity of the final Cu product of Equation 15b was not verified, but is believed to be analogous to the common copper(1) starting material [Cu(CH₃CN)₄]⁺. Thus, Equation 16c, exhibits an equilibrium constant of 1.2×10¹³ M⁻².

[Cu(dtbp)₂]⁺+CH₃NC [symbol] [Cu(dtbp)(CH₃NC)]⁺+dtbp (Eq. 15a)

[Cu(dtbp)(CH₃NC)]⁺+3CH₃NC+dtbp [symbol] [Cu(CH₃NC)₄]⁺+2dtbp (Eq. 15b)

[Cu(dtbp)₂]⁺+4CH₃NC [symbol] [Cu(CH₃NC)₄]⁺+2dtbp (Eq. 15c)

Photophysical and electrochemical attributes of [Cu(dtbp)₂][B(C₆F₅)₄]. The photophysical measurements revealed a quantum yield on par with and an excited state lifetime longer than those of [Ru(bpy)₃]²⁺. The exceptional steric constraints in complex 1 weaken the metal-ligand bonding, which in turn afforded a unique type of reactivity. One of the chelating dtbp ligands is readily replaced by strongly donating, monodentate ligands such as acetonitrile and CO. The unique combination of excellent photophysical properties and ligand displacement reactivity renders Complex 1 attractive for use in sensors, molecular machines or photoelectronic devices.

Bulky Cu(I) Complexes for Use in Photovoltaic Cells

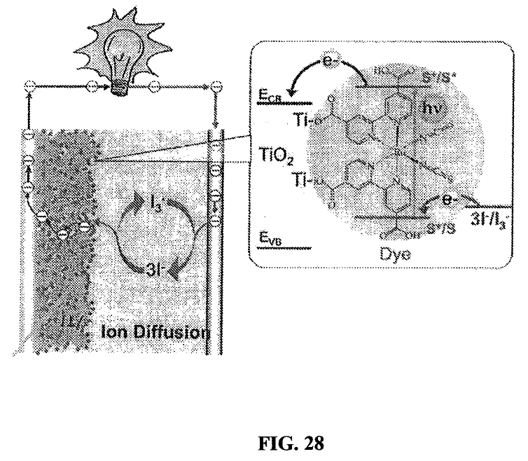

This example demonstrates that luminescent Cu(I) complexes, based on the ligand 2,9-di-t-butyl-1,10-phenanthroline, can serve as effective sensitizers for Gratzel-type dye sensitized solar cells. The solar cells are based on the sensitization of mesoscopic oxide films by dyes or quantum dots. These systems have already reached conversion efficiencies exceeding 11%. The underlying fundamental processes of light harvesting by the sensitizer, heterogeneous electron transfer from the electronically excited chromophore into the conduction band of the semiconductor oxide, and percolative migration of the injected electrons through the mesoporous film to the collector electrode as also shown in FIG. 28. These solar cells have now also been used in outdoor applications.

FIG. 28 depicts a schematic drawing showing the currently used embodiment of the Dye-Sensitized Photovoltaic Cells (DSC) utilizing cis-Ru(SCN)₂L₂ (L=2,2′-bipyridyl-4,4′-dicarboxylate). It employs dye-derivatized TiO₂ nanocrystals as light-harvesting units. The sensitizer is cis-Ru(SCN)₂L₂ (L=2,2′-bipyridyl-4,4′-dicarboxylate). The redox system employed to regenerate the dye and transport the positive charges to the counter electrode is the iodide/triiodide couple dissolved in an organic electrolyte or in a room-temperature ionic liquid.

In this DSC, the light harvesting unit comprises TiO₂ nanoparticles. In this system described by Gratzel, the cis-Ru(SCN)₂L₂ (L=2,2′-dipyridyl-4,4′-dicarboxylate) may be substituted with the appropriately modified copper(I) complex L₁L₂CuX. However, it is important that the ligands be modified to be successful in the method of the present invention For instance, the ligands need at least one or two COOH groups on the “back” side to attach to the TiO₂ nanoparticles. At least one of the ligands in the complex must have the COOH modification. Said modification may be done via routine experiments known to one or ordinary skill in the art, as described further below.

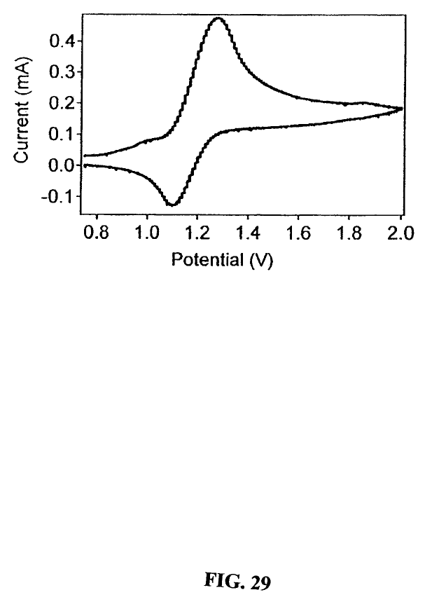

Electrochemical Analysis. The reduction potential of complex 1 (0.1 M) in CH₂Cl₂ solution was measured by cyclic voltammetry (FIG. 29). The voltammetric behavior of complex 1 was not ideal: in a single cycle the forward and reverse currents were not the same (iₚₐ/i_{pc}=0.75, where iₚₐ and i_{pc} are the intensities of the anodic and cathodic peak, respectively), and the current flow in both sweep directions decreased upon a second cycle. These observations suggest that the complex degraded during the course of the experiment. Reasonable behavior was obtained at a sweep rate of 50 mV/sec. The separation between the anodic and cathodic peaks, 180 mV, was within the 100-200 mV separation that has been observed for others of this class of complexes. The apparent E_{1/2} of 120 mV (vs. Ag|AgCl) was assigned to the Cu^{2+/+} couple.

An estimate of the reduction potential of the excited state may be calculated from the emission spectral data and the ground state reduction potential. The maximum free energy of an emissive state (ΔGₑₛ, eV) may be determined by extending a tangent from the high-energy side of the emission spectrum to the energy axis. The value for ΔGₑₛ of Complex 1 obtained using this method and those reported for related complexes in the literature, are listed in Table 9.

TABLE 9
The Cu^{2+/+} potentials of selected [Cu(R₂Phen)₂]⁺ complexes
and their derived excited-state reduction potentials. The first four
complexes are listed in order of decreasing bulk of the alkyl ligand.

| Complex | E(Cu^{2+/+}),^{a} V | ΔGₑₛ, eV | E(Cu^{2+/+}*),^{b} V |
|---|---|---|---|
| [Cu(dtbp)₂]⁺ | 0.70 | 2.36 | −1.66 |
| [Cu(dsbp)₂]⁺ | 0.68 | 2.21 | −1.53 |
| [Cu(dbp)₂]⁺ | 0.61 | 2.14 | −1.53 |
| [Cu(dmp)₂]⁺ | 0.50 | 2.04 | −1.54 |
| [Cu(bfp)₂]⁺ | ~11 | 2.14 | −1.04 |

Abbreviations:
bfp, 2,9-bis(trifluoromethyl)-1,10-phenanthroline;
dtbp, 2,9-di-tert-butyl-1,10-phenanthroline;
dsbp, 2,9-sec-butyl-1,10-phenanthroline;
dbp, 2,9-dibutyl-1,10-phenanthroline;
dmp, 2,9-dimethyl-1,10-phenanthroline.
^{a}All values listed were measured versus ferrocene (Fc^{+/0}) in CH₂Cl₂.
^{b}E(Cu^{2+/+}*) = E(Cu^{2+/+}) − ΔGₑₛ; calculated versus ferrocene (Fc^{+/0}).

The excited state potential [Cu²⁺ (R₂Phen)₂] (Complex 13) may be estimated from ΔGₑₛ, which is the maximum energy difference between photoexcited Complex 13 and ground Complex 13, and E_{1/2}, which measures the energy difference. The difference between E_{1/2} and ΔGₑₛ is then the potential for the reduction process. Using this method, the excited state reduction potential of Complex 1 was estimated to be −1.66 V versus ferrocene^{+/0}.

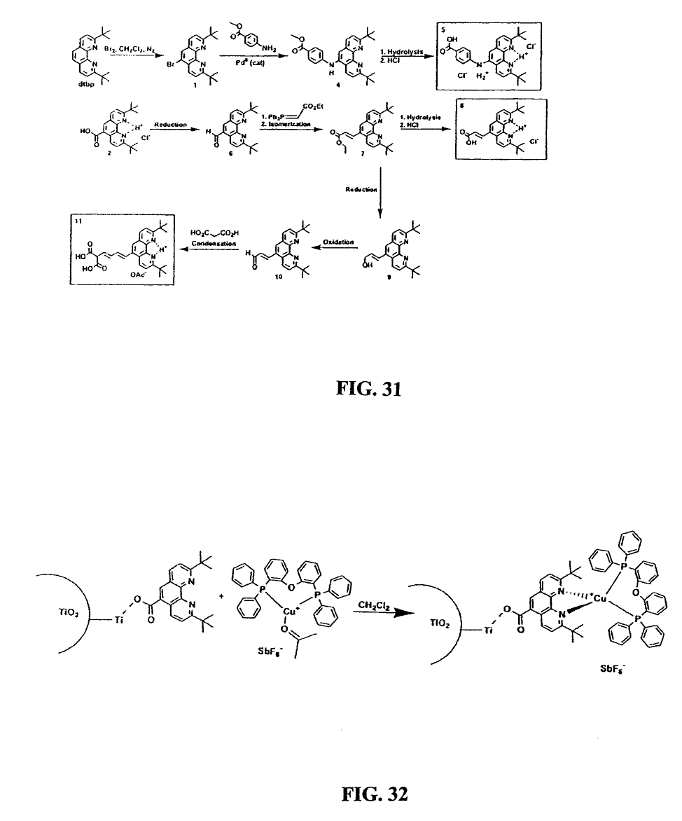

To optimize electron delivery into the conduction band of the TiO₂ substrate, it may be necessary to functionalize a ligand to provide a point of coordinate covalent attachment to the Ti atoms of the substrate. The method to provide such an attachment is shown in FIG. 31.

Modification of phenanthroline-derived ligand for coordinate covalent attachment to metal-oxide semiconductor surfaces is now described. A carboxylate functionality will be added to the dtbp ligand to provide a site of coordinate covalent attachment to the surface Ti atoms of the substrate. This is precisely the method used by Gratzel and co-workers to append tris-bipyridyl ruthenium(II) complexes to the substrate. As was done for the ruthenium sensitizers, the position of the carboxylate tether relative to the phenanthroline will be varied, maintaining conjugation to the ring in order to optimize the potential for electron delivery into the substrate. A key intermediate in the functionalization of dtbp is the mono-brominated species 1; the procedure for synthesis of this compound was adapted from Eggert et al., who used the method to prepare 5-bromo-2,9-dimethyl-1,10-phenanthroline. This method has been applied to dtbp to produce an approximately 50% yield.

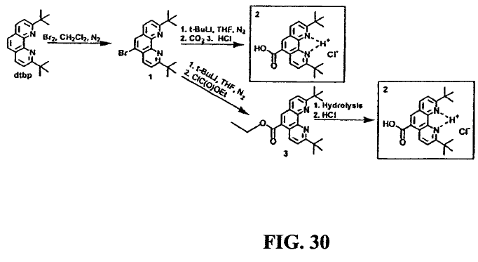

To prepare the appended carboxylate functionality with a minimal tether length 2, two distinct routes are proposed (FIG. 30). To create a longer and fairly rigid tether an intervening phenyl group will be used; the synthetic procedure shown to make Complex 5 follows the method of Eggert et al. for functionalization of the analogous 2,9-dimethyl-1,10-phenanthroline. Molecules with longer, conjugated tethers, 8 and 11, will be prepared by the same strategy used to functionalize a zinc porphyrin for attachment to TiO₂.

In one strategy the dye will be assembled stepwise on the TiO₂ surface, as illustrated schematically for a representative set of ligands in FIG. 32. First the carboxylate functionalized dtbp ligand of choice will be reacted with the substrate. Second, a three coordinate Cu(I) solvato complex, bearing the auxiliary ligand of choice, will be added to create the dye complex. The dye-functionalized TiO₂ will be tested in a Gratzel-type DSC device, illustrated in FIG. 23, and constructed according to the Gratzel protocol.

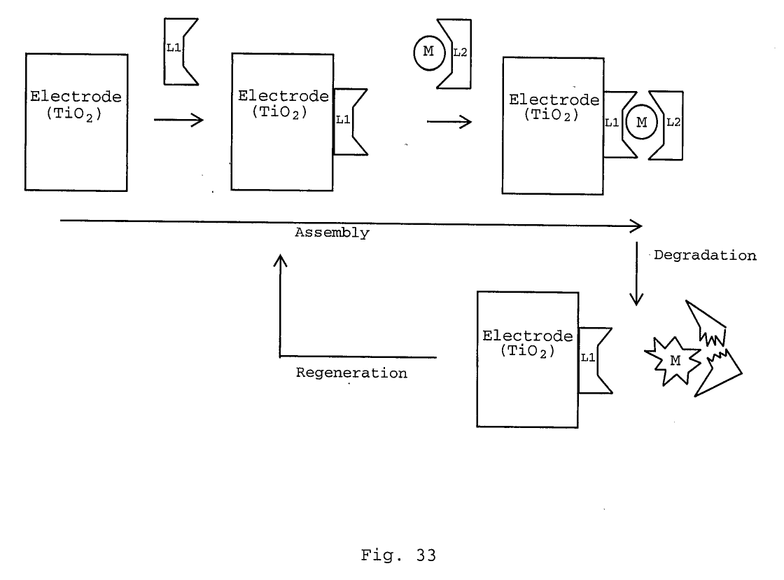

Assembly of Cu(I)-based sensitizer dyes on TiO₂ electrodes is now described. One advantageous feature of the present Cu(I) complex synthesis method is that the dye sensitizer complex can be prepared stepwise on the surface of the metal oxide (FIG. 33). Once the functionalized phenanthroline ligand has been attached to the TiO₂ surface, a three coordinate Cu(I) solvato complex will be added to create the dye complex. In this second step, auxiliary ligand on the solvato complex may be varied, such that the resulting complex on the surface will have distinct absorption properties. A mixture of complexes may be added, such that an ensemble of dye molecules with different absorption properties will be created, maximizing the wavelength range over which light will be absorbed. The three coordinate Cu(I) solvato complexes, bearing various auxiliary ligands designed to extend the wavelength range over which the dye complexes absorb, may be synthesized independently using the oxidation-based route.

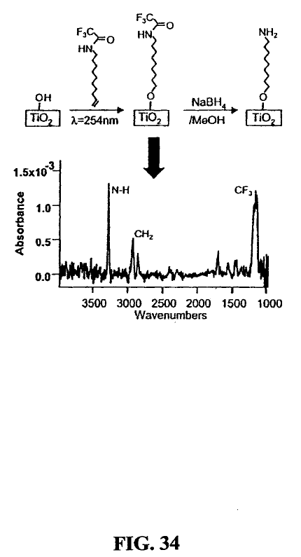

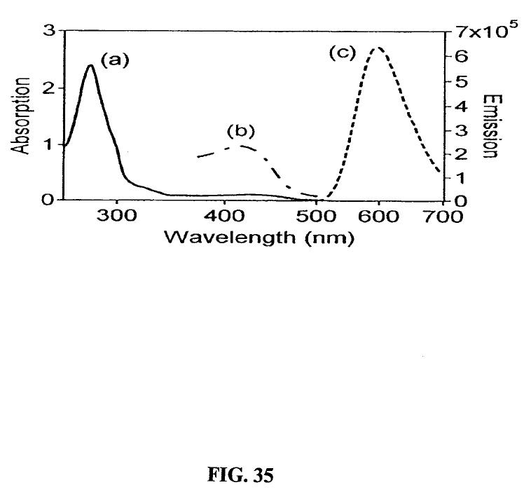

The basis for this method is shown in FIG. 34. Several auxiliary ligands that may be used are shown. It is important to note that all the Cu(I) complexes prepared are remarkably resistant to oxidation, including reaction with molecular oxygen. In the solid state, [Cu(dtbp)(acetone)]⁺ appears to be indefinitely stable in the air. [Cu(dtbp)₂]⁺ is even more reluctant to react with oxygen, and with a reduction potential of +1.2 V vs. Ag|AgCl, this complex is not prone to oxidation.

### Bulky Cu(I) Complexes as a Solid-State Lighting Device

A light-emitting electrochemical cell (LEEC) is a thin-film light-emitting electrochemical cell in which the emissive layer is an organic compound. LEEC technology is intended primarily as picture elements in practical display devices. These devices promise to be much less costly to fabricate than traditional liquid crystal displays. When the emissive electroluminescent layer is polymeric, varying amounts of LEECs can be deposited in rows and columns on a screen using simple “printing” methods to create a graphical color display, for use as television screens, computer displays, portable system screens, and in advertising and information board applications. LEECs may also be used in lighting devices. LEECs are available as distributed sources while the inorganic liquid crystal displays are point sources of light.

A LEEC works on the principle of electroluminescence. The key to the operation of a LEEC is an organic luminophore. An exciton, which consists of a bound, excited electron and hole pair, is generated inside the emissive layer. When the exciton's electron and hole combine, a photon can be emitted. A major challenge in LEEC manufacture is tuning the device such that an equal number of holes and electrons meet in the emissive layer. This is difficult because, in an organic compound, the mobility of an electron is much lower than that of a hole.

An exciton can be in one of two states, singlet or triplet. Only one in four excitons is a singlet. The materials currently employed in the emissive layer are typically fluorophors, which can only emit light when a singlet exciton forms, which reduces the LEEC's efficiency.

By incorporating transition metals into a small-molecule LEEC, the triplet and singlet states can be mixed by spin-orbit coupling, which leads to emission from the triplet state. However, this emission is always redshifted, making blue light more difficult to achieve from a triplet excited state. Triplet emitters can be four times more efficient than LEEC technology.

To create the excitons, a thin film of the luminophore is sandwiched between electrodes of differing work functions. Electrons are injected into one side from a metal cathode, while holes are injected in the other from an anode. The electron and hole move into the emissive layer and can meet to form an exciton. Mechanisms and details of exciton formation are well known and established in literature.

Derivatives of poly(p-phenylene vinylene) (PPV) and poly(fluorene), are commonly used as polymer luminophores in LEECs. Indium tin oxide is a common transparent anode, while aluminum or calcium are common cathode materials. Other materials are added between the emissive layer and the cathode or the anode to facilitate or hinder hole or electron injection, thereby enhancing the LEEC efficiency.

The properties of [Cu(dtbp)₂]⁺ extend established trends in absorption and emission characteristics of bis(phenanthroline)Cu(I) complexes. Table 10 lists optical characteristics of selected bis(phenanthroline)Cu(I) complexes with varied alkyl substituents at the 2 and 9 positions of the ligand and organized in order of decreasing emission lifetime and decreasing quantum yield. The lifetime and quantum yield correlate with the apparent steric bulk of the ligand: [Cu(dtbp)₂]⁺ is the bulkiest of this series and exhibits the longest observed lifetime and highest quantum yield. [Cu(dtbp)₂]⁺ also exhibits the smallest difference between absorption and emission wavelengths in this class. Since the emitting state is believed to be a triplet, the magnitude of this shift is related to the energy difference between the two excited states (¹MLCT and ³MLCT) and to the geometric reorganization between the ground and excited states. The 174-nm difference observed in [Cu(dtbp)₂]⁺ implies that there is either a smaller energy difference between the two excited states, a smaller reorganization between ground and excited states, or both, relative to other complexes of this series. The oscillator strength of [Cu(dtbp)₂]⁺, with an ε of 3100 M⁻¹ cm⁻¹, is among the smallest of this series presumably due to the poorer overlap between the metal and ligand orbitals that is a consequence of the elongated Cu—N bonds.

TABLE 10
Photophysical characteristics of bis(2,9-dialkyl-phenanthroline)Cu(I) complexes. The complexes
are listed in order of decreasing excited state emission lifetime and quantum yield.

| Complex | λ_{Abs} (nm) | ε (L mol⁻¹ cm⁻¹) | λ_{Em} (nm) | λ_{Em} − λ_{Abs} (nm) | τ (ns) | φₑₘ × 10³ |
|---|---|---|---|---|---|---|
| [Cu(dtbp)₂]⁺ | 425 | 3100 | 599 | 174 | 3260 | 56 |
| [Cu(dtbp)(dmp)]⁺ | 440 | 7000 | 646 | 206 | 730 | 10 |
| [Cu(dsbp)₂]⁺ | 455 | 6600 | 690 | 235 | 400 | 4.5 |
| [Cu(dipp)₂]⁺ | 445 | 7500 | 650 | 205 | 365 | 4.0 |
| [Cu(dnpp)₂]⁺ | 449 | 5700 | 715 | 265 | 260 | 1.6 |
| [Cu(dbp)₂]⁺ | 457 | 7000 | 725 | 268 | 150 | — |
| [Cu(dmp)₂]⁺ | 454 | 7800 | 730 | 276 | 90 | 0.23 |

Abbreviations:
dtbp, 2,9-di-tert-butyl-1,10-phenanthroline;
dmp, 2,9-dimethyl-1,10-phenanthroline;
dsbp, 2,9-sec-butyl-1,10-phenanthroline;
dipp, 2,9-di-isopropyl-1,10-phenanthroline;
dnpp, 2,9-dineopentyl-1,10-phenanthroline;
dbp, 2,9-butyl-1,10-phenanthroline

Photophysical Attributes of Complex 1. The absorption spectrum, emission spectrum and excitation profile of Complex 1 are shown in FIG. 24. The absorption features are typical of bis(phenanthroline)copper(I) complexes, including intense π→π* ligand absorption (275 nm) and less intense dπ643 π* MLCT absorption (425 nm). The magnitude of the extinction coefficient of the MLCT transition (Table 11) is on the order expected for a d π→π* transition. Excitation into this absorption band produces a broad emission band, with a maximum at 599 nm, typical of emission from an MLCT excited state. The 425 nm feature is absent from the spectrum of the dtbp ligand, supporting the assignment of this band to the MLCT absorption in the complex.

TABLE 11
Photophysical characteristics of [Cu(R₂Phen)₂]⁺ complexes in
CH₂Cl₂ solution and comparison with those of [Ru(bpy)₃]⁺.
The data for [Ru(bpy)₃]⁺ are presented as a range because they were
collected in a variety of solvents.

| | | ε_{λmax}, L mol⁻¹ | | λ_{Abs}-λ_{Em}, | | | kᵣ | kₙᵣ |
|---|---|---|---|---|---|---|---|---|
| Complex | λ_{Abs}, nm | cm⁻¹ | λ_{Em}, nm | eV | τ, ns | φₑₘ × 10³ | (×10³, s⁻¹) | (×10⁷, s⁻¹) |
| [Ru(bpy)₃]²⁺ | 446-454 | ~14000 | 580-630 | 0.617-0.799 | 480-1250 | 28-110 | — | — |
| [Cu(dtbp)₂]⁺ | 425 | 3100 | 599 | 0.847 | 3260 | 56 | 17.18 | 0.03 |
| [Cu(dtbp)(dmp)]⁺ | 440 | 7000 | 646 | 0.899 | 730 | 10 | 13.70 | 0.14 |
| [Cu(dsbp)₂]⁺ | 455 | 6600 | 690 | 0.928 | 400 | 4.5 | 11.25 | 0.25 |
| [Cu(dipp)₂]⁺ | 445 | 7500 | 650 | 0.879 | 365 | 4.0 | 10.90 | 0.27 |
| [Cu(dnpp)₂]⁺ | 449 | 5700 | 715 | 1.027 | 260 | 1.6 | 6.15 | 0.38 |
| [Cu(dbp)₂]⁺ | 457 | 7000 | 725 | 1.003 | 150 | 0.9 | 6.00 | 0.66 |
| [Cu(dmp)₂]⁺ | 454 | 7800 | 730 | 1.033 | 90 | 0.23 | 2.71 | 1.18 |
| [Cu(dpp)₂]⁺ | 440 | 3800 | 690 | 1.021 | 280 | 2.5 | 8.93 | 3.56 |
| [Cu(bfp)₂]⁺ | 462 | 10900 | 665 | 0.819 | 165 | 3.3 | 24.00 | 0.60 |
| [Cu(xop)₂]⁺ | 452 | 3000 | 673 | 0.901 | 149 | 1.0 | 6.71 | 0.67 |

Abbreviations:
dtbp, 2,9-di-tert-butyl-1,10-phenanthroline;
dmp, 2,9-dimethyl-1,10-phenanthroline;
dsbp, 2,9-sec-butyl-1,10-phenanthroline;
dipp, 2,9-di-isopropyl-1,10-phenanthroline;
dnpp, 2,9-dineopentyl-1,10-phenanthroline;
dbp, 2,9-butyl-1,10-phenanthroline;
xop, 2(2-methylphenyl)-9-(2,6-dimethylphenyl)-1,10-phenanthroline;
dpp, 2,9-diphenyl-1,10-phenanthroline;
bfp, 2,9-bis(trifluoromethyl)-1,10-phenanthroline

Complex 1 exhibits the longest lifetime and highest quantum yield for this class of compounds, substantially larger than those observed for [Cu(dtbp)(dmp)]⁺. MLCT-derived emission from 1 is characterized by a lifetime (τ) of 3260 ns and an estimated quantum yield (φ) of 5.6 (±0.4) % (5.6×10⁻²). The improvement in the emission characteristics of Complex 1, relative to those of [Cu(dtbp)(dmp)]⁺, appears to be due to a reduction in the non-radiative relaxation rate by almost a full order of magnitude. The calculated radiative relaxation rate (kᵣ= [symbol] τ) for Complex 1 is 1.9 (±0.2)×10⁴ s⁻¹ while the non-radiative relaxation rate (kₙᵣ=(1− [symbol] [symbol] is 3.0 (±0.2)×10⁵ s⁻¹. The quantum yield of Complex 1 in CH₂Cl₂ is approximately 50% larger than that of [Ru(bpy)₃][(PF₆)₂] in water. Direct comparison of the MLCT-derived emission intensity of Complex 1 with that of [Cu(dtbp)(dmp)](BF₄), under the same solution conditions, indicate that the quantum yield of Complex 1 is approximately five times larger (FIG. 25).

Those skilled in the art will recognize, or be able to ascertain using no more then routine experimentation, numerous equivalents to the specific compounds, ligands methods, assays and reagents described herein. Such equivalents are considered to be within the scope of this invention and covered by the following claims. All publications, patents, and patent applications cited herein are hereby incorporated by reference in their entirety for all purposes.

What is claimed is:

1. A copper(I) complex having the formula L₁L₂CuX wherein X is a negatively charged ion and the ligands L₁ and L₂ are independently selected from 2,9-di-tert-butyl-1,10-phenanthroline, 2,9-di-tert-butyl-4,7-diphenyl-1,10-phenanthroline, and 2,9-di-2,4,6-trimethylphenyl-1,10-phenanthroline.

2. The copper(I) complex of claim 1 wherein the ion X is selected from SO₃CF₃⁻, BF₄⁻, SbF₆⁻, B(C₆F₅)₄⁻, PF₆⁻ and ClO₄⁻.

3. A method of synthesizing a copper(I) complex comprising the steps of:
(a) mixing a ligand L and AgX in a molar ratio of at least 2:1 and solid copper in a polar solvent to result in a (L)₂CuX complex; and
(b) separating the (L)₂CuX complex from the reaction of step (a), wherein X is a negatively charged ion.

4. The method of claim 3 wherein the polar solvent is selected from acetone, ethanol and tetrahydrofuran.

5. The method of claim 3 wherein the ion X is selected from SO₃CF₃⁻, BF₄⁻, SbF₆⁻, B(C₆F₅)₄⁻, PF₆⁻ and ClO₄⁻.

6. The method of claim 3 wherein the ligand L is selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline.

7. A method of synthesizing a copper(I) complex comprising the steps of:
(a) mixing a ligand L₁ with AgX in a molar ratio of about 1:1 and solid copper in a polar solvent to result in a L₁CuX complex;
(b) isolating the resulting L₁CuX complex; and
(c) adding about one molar equivalent of ligand L₂ to complex L₁CuX in a non-polar solvent, resulting in a L₁L₂CuX complex; and
(d) separating the L₁L₂CuX complex from the reaction of step (c), wherein X is a negatively charged ion.

8. The method of claim 7 wherein the ligand L₁ has the same chemical structure as ligand L₂.

9. The method of claim 7 wherein the ion X is selected from SO₃CF₃⁻, BF₄⁻, SbF₆⁻, B(C₆F₅)₄⁻, PF₆⁻ and ClO₄⁻.

10. The method of claim 7 wherein the polar solvent of step (a) is selected from acetone, ethanol and tetrahydrofuran.

11. The method of claim 7 wherein the non-polar solvent of step (c) is dichloromethane.

12. The method of claim 7 wherein ligands L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline.

13. A method of detecting a target molecule in a system having or suspected of having the target molecule, the method comprising the steps of:
(a) contacting a luminescent copper(I) L₁L₂CuX complex with the system having or suspected of having the target molecule;
(b) binding the target molecule to the copper(I) L₁L₂CuX complex, wherein the target molecule has a binding constant for Cu(I) that is greater than a Cu(I) binding constant possessed by at least one of the ligands L₁ or L₂; and
(c) detecting the presence of the target molecule by measuring a reduction or increase in luminescence of the copper(I) complex.

14. The method of claim 13 wherein the target molecule is selected from CO, CH₃CN, C₂H₄, CH₃NC, C₂H₂, NO and O₂.

15. The method of claim 13 wherein the ligands L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline.

16. The method of claim 13 wherein X is a negatively charged ion.

17. The method of claim 16 wherein the ion X is selected from SO₃CF₃⁻, BF₄⁻, SbF₆⁻, B(C₆F₅)₄⁻, PF₆⁻ and ClO₄⁻.

18. A dye-sensitized photovoltaic cell comprising a light harvesting unit and a sensitizer, wherein the sensitizer is a copper(I) L₁L₂CuX complex, wherein L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline; and wherein X is a negatively charged ion.

19. The photovoltaic cell of claim 18 wherein the ion X is selected from SO₃CF₃⁻, BF₄⁻, SbF₆⁻, B(C₆F₅)₄⁻, PF₆⁻ and ClO₄⁻.

20. The photovoltaic cell of claim 18, wherein the light harvesting unit comprises TiO₂ nanoparticles.

21. A light-emitting electrochemical cell having an emissive layer, the cell comprising a copper(I) L₁L₂CuX complex, wherein L₁ and L₂ are independently selected from: 1,10-phenanthroline; 2,9-di-neo-pentyl-1,10-phenanthroline; 2,9-di-sec-butyl-1,10-phenanthroline; 2,9-di-tert-butyl-1,10-phenanthroline; 2,9-di-tert-butyl-4,7-diphenyl-1,10-phenanthroline; 2,9-dimethyl-1,10-phenanthroline; 2,9-diphenyl-1,10-phenanthroline; 2,9-dimethyl-4,7-diphenyl-1,10-phenanthroline; and 2,9-bis-trifluoromethyl-1,10-phenanthroline; and wherein X is a negatively charged ion.

22. The electrochemical cell of claim 21 wherein the ion X is selected from SO₃CF₃⁻, BF₄⁻, SbF₆⁻, B(C₆F₅)₄⁻, PF₆⁻ and ClO₄⁻.

23. A crystalline form of copper(I) complex having unit cell dimensions of about:
a=11.8246 Å; b=17.8044 Å; and c=27.1111 Å;
a=14.906 Å; b=15.188 Å; and c=16.754 Å;
a=14.7039 Å; b=25.883(3) Å, and c=16.7036(16) Å;
a=14.75449(9) Å, b=15.1383(9) Å, and c=17.9557(11) Å; or
a=12.1995(5) Å, b=13.6275(6) Å, and c=14.4027(6) Å.

24. The crystalline form of copper (I) complex of claim 23 wherein the copper(I) complex is [(2,9-di-tert-butyl-1,10-phenanthroline)₂Cu][B(C₆F₅)₄].
